(19) United States
(12) Patent Application Publication
(10) Pub. No.: US 2022/0008112 A1
(43) Pub. Date: Jan. 13, 2022

(54) ULTRASOUND TRANSDUCER AND SYSTEM FOR SKIN TREATMENTS

(71) Applicant: Sofwave Medical Ltd., Yokneam (IL)

(72) Inventors: Ariel SVERDLIK, Tel-Aviv (IL); Shimon ECKHOUSE, Haifa (IL); Assaf GELSTEIN, Haifa (IL)

(73) Assignee: Sofwave Medical Ltd., Yokneam (IL)

(21) Appl. No.: 17/485,860

(22) Filed: Sep. 27, 2021

Related U.S. Application Data
(63) Continuation of application No. PCT/IL2020/050368, filed on Mar. 26, 2020.
(60) Provisional application No. 62/824,503, filed on Mar. 27, 2019.

(51) Int. Cl. A61B 18/02

(52) U.S. Cl. CPC A61B 18/0206; A61B 2018/00458

(57) ABSTRACT

A method for treating skin, including:
delivering one or more pulses of non-converging ultrasonic energy through a surface area size in a range of 3 mm²-7 mm², wherein each pulse having an intensity in a range of 5 W/cm²-60 W/cm² and a time duration in which the ultrasonic energy is actively transmitted in a range of 1-10 seconds per pulse, wherein the non-converging ultrasonic energy is delivered from a fixed position to one or more skin regions having a maximal surface area size in a range of 5 cm²-100 cm².

### RELATED APPLICATIONS

This application is a Continuation of PCT Patent Application No. PCT/IL2020/050368 having International filing date of Mar. 26, 2020, which claims the benefit of priority under 35 USC § 119(e) of U.S. Provisional Patent Application No. 62/824,503 filed Mar. 27, 2019.

PCT Patent Application No. PCT/IL2020/050368 is also related to PCT Patent Application No. PCT/IL2017/050638, filed on Jun. 6, 2017.

The contents of the above applications are all incorporated by reference as if fully set forth herein in their entirety.

### FIELD AND BACKGROUND OF THE INVENTION

The present invention, in some embodiments thereof, relates to treating tissue using ultrasound energy and, more particularly, but not exclusively, to an ultrasonic transducer and applicator for skin treatments.

US Publication number U.S. Pat. No. 6,595,934 B1 discloses “A method of skin rejuvenation by thermal ablation using high intensity focused ultrasound energy includes the steps of positioning an ultrasound emitting member adjacent an external surface of the skin, emitting ultrasound energy from the ultrasound emitting member into the skin, focusing the ultrasound energy in the skin, ablating the skin with the focused ultrasound energy to form an ablated tissue area below the external surface of the skin containing unablated tissue of the skin and a plurality of lesions at which the tissue of the skin is ablated, and removing the ultrasound emitting member from adjacent the external surface of the skin. The lesions cause collagen production by the skin to be stimulated. The lesions can begin and end at predetermined depths beneath the external surface of the skin so that the epidermis and the deep layer of the dermis are not damaged.”

### SUMMARY OF THE INVENTION

Some examples of some embodiments of the invention are listed below:
Example 1. A method for treating skin, comprising:

delivering one or more pulses of non-converging ultrasonic energy through a surface area size in a range of 3 mm²-7 mm², wherein each pulse having an intensity in a range of 5 W/cm²-60 W/cm² and a time duration in which said ultrasonic energy is actively transmitted in a range of 1-10 seconds per pulse, wherein said non-converging ultrasonic energy is delivered from a fixed position to one or more skin regions having a maximal surface area size in a range of 5 cm²-100 cm².
Example 2. A method according to example 1, wherein said each pulse has an energy intensity in a range of 15 W/cm²-30 W/cm².
Example 3. A method according to any one of the previous examples, comprising:

cooling an external surface of the one or more skin regions during and/or following said delivering.
Example 4. A method according to example 3, wherein said cooling comprises cooling said external surface to maintain a temperature of the epidermis between 5-40 degrees Celsius.
Example 5. A method according to any one of the previous examples, comprising:

heating by said delivered non-converging ultrasonic energy one or more tissue layers located at a depth of 0.5 mm-3 mm from an external surface of the skin to a temperature of at least 45 degrees Celsius.
Example 6. A method according to any one of the previous examples, wherein said one or more skin regions are located at facial or neck regions.
Example 7. A method according to any one of the previous examples, comprising:

identifying one or more regions in said skin area which include one or more nerves located at a depth in a range of 0.5 mm-3 mm from an external surface of the skin prior to said delivering, and wherein said delivering comprises delivering said non-converging ultrasonic energy to a skin area that do not include said one or more regions.
Example 8. A method according to any one of the previous examples, wherein said delivering comprises delivering said one or more pulses of non-converging ultrasonic energy to said one or more skin regions in at least two repetitions with a time difference of at least 30 seconds between the repetitions.
Example 9. A method according to any one of the previous examples, wherein said delivering comprises delivering said non-converging ultrasonic energy for a time period long enough to have a reduction of at least one point in a wrinkles severity scale comprising one or more of the Wrinkle Severity Rating Scale (WSRS), the Glogau scale, the Fitzpatrick wrinkle scale, the Fitzpatrick wrinkle score, and/or the Fitzpatrick Wrinkle and Elastosis Scale (FWES), at least a week after said delivering.
Example 10. A method for treating skin, comprising:

delivering one or more pulses of non-converging ultrasonic energy, wherein each pulse has an ultrasound intensity in a range of 15 W/cm²-30 W/cm² and a time duration in which said ultrasonic energy is actively transmitted in a range of 2-6 seconds per pulse.
Example 11. A method for reducing a severity of wrinkles, comprising:

selecting one or more parameter values of non-converging ultrasonic energy to be delivered to at least one region of skin tissue which are suitable to reduce at least one point of a wrinkles severity scale after at least one week following a delivery of ultrasonic energy in said region, wherein said wrinkles severity scale comprises one or more of the Wrinkle Severity Rating Scale (WSRS), the Glogau scale, the Fitzpatrick wrinkle scale, the Fitzpatrick wrinkle score, and/or the Fitzpatrick Wrinkle and Elastosis Scale (FWES);

delivering said non-converging ultrasonic energy with said selected one or more parameters values to said at least one region of skin tissue.
Example 12. A method according to example 11, wherein said selecting comprises reading said one or more parameter values from a table in a memory.
Example 13. A method according to any one of examples 11 or 12, wherein said selecting comprises selecting said one or more parameter values according to one or more parameters of said skin region.
Example 14. A method according to example 13, wherein said skin region parameters comprise one or more of tissue type of said skin, tissue composition in said skin region, fat tissue content in said skin region, location of said skin region, presence of nerves in or near said skin region.
Example 15. A method according to any one of examples 11 to 14, wherein said one or more parameter values comprise one or more pulses of ultrasonic energy each having an energy intensity in a range of 15 W/cm²-30 W/cm² and a time duration in which said ultrasonic energy is actively delivered in a range of 2-6 seconds per pulse through a surface area size in a range of 3 mm²-7 mm², and wherein said delivering comprises delivering said non-converging ultrasonic energy with said selected one or more parameter values to cover an external surface of said skin tissue having a surface area in a range of 5 cm²-100 cm².
Example 16. A method according to any one of examples 11 to 15, comprising:

cooling an external surface of said skin tissue during and/or following said delivering to maintain a temperature of the epidermis between 5-40 degrees Celsius.
Example 17. A method according to any one of examples 11 to 16, comprising heating by said delivered non-converging ultrasonic energy one or more tissue layers located at a depth of 0.25 mm-5 mm from said external surface of the skin to a temperature of at least 45 degrees Celsius.
Example 18. A method according to any one of examples 11 to 17, comprising: identifying one or more regions of skin tissue which include nerves located at a distance of up to 5 cm from an external surface of the skin prior to said delivering, and wherein said delivering comprises delivering said non-converging ultrasonic energy to said at least one region of skin tissue that do not include said one or more identified regions.
Example 19. A method according to any one of examples 11 to 18, wherein said region of skin tissue is located in facial or neck regions.
Example 20. A system for treating wrinkles, comprising:

an ultrasound applicator comprising;

a plurality of ultrasound transducers configured to generate non-converging ultrasonic energy, wherein each of said ultrasound transducers comprises an active surface with an area surface size in a range of 3 mm²-7 mm²;

housing shaped and sized to place at least some of said ultrasound transducers in contact with an external surface of skin tissue;

a control console connected to said ultrasound applicator, comprising:

a memory for storing parameter values of said ultrasonic energy;

a control circuitry configured to signal said ultrasound transducers to generate one or more pulses of ultrasonic energy, wherein each of said one or more pulses has an energy intensity in a range of 5 W/cm2-60 W/cm² and a time duration in which said ultrasonic energy is actively transmitted in a range of 2-6 seconds per pulse.
Example 21. A system according to example 20, wherein each of said one or more pulses has an energy intensity in a range of 15 W/cm²-30 W/cm².
Example 22. A system according to any one of examples 20 or 21, wherein said applicator comprises a cooling module configured to cool said plurality of ultrasound transducers and/or regions of said applicator placed in contact with the skin.
Example 23. A system according to example 22, wherein said cooling module comprises one or more thermo electric coolers (TECs) each having a cold surface and a hot surface, wherein said cold surface is configured to cool said ultrasound transducers and/or regions between said ultrasound transducers.
Example 24. A system according to example 23, wherein said cooling module comprises a cooling fluid chamber comprising cooling fluid, wherein said cooling fluid chamber is configured to cool said hot surface of said one or more TECs.
Example 25. A system according to example 24, comprising an elongated cable connecting said applicator and said control console, wherein said elongated cable comprises electrical wiring and one or more cooling fluid flow paths shaped and sized to allow circulation of said cooling fluid between said cooling fluid chamber and said control console.
Example 26. A cosmetic method for skin treatments, comprising:

storing in a memory, parameters related to skin tissue properties and/or composition at a selected treatment target in a specific subject, and values of at least one treatment parameter of a cosmetic unfocused ultrasound treatment;

automatically adjusting by a control circuitry connected to said memory said stored values of said at least one treatment parameter based on said stored skin tissue-related indication;

signaling at least one ultrasound transducer to emit unfocused ultrasound energy to said selected treatment target according to said automatically adjusted values.
Example 27. A method according to example 26, comprising:

generating a user-specific profile including said skin tissue-related indications and said adjusted values, and wherein said storing comprises storing said user-specific profile in said memory.
Example 28. A method according to any one of examples 26 or 27, wherein said storing comprises storing in said memory evaluation results of an unfocused ultrasound treatment delivered to said subject, and wherein said automatically adjusting comprises automatically adjusting said values based on said stored evaluation results.
Example 29. A method according to any one of examples 26 to 28, wherein said skin tissue-related indications comprise at least one of skin tissue composition and/or location of a selected target tissue volume at said selected treatment target.
Example 30. A method according to any one of examples 26 to 29, wherein said skin tissue-related indications comprises at least one of wrinkles length, wrinkles depth, and/or wrinkles density at said selected treatment target.
Example 31. A method according to any one of examples 26 to 30, wherein said at least one treatment parameter comprises ultrasound energy intensity, duration of an ultrasound energy pulse, number of transducers, temperature and/or duration of skin surface cooling.
Example 32. A cosmetic method for skin treatments, comprising:

storing in a memory one or more safety indications related to a specific subject, and values of at least one treatment parameter of a cosmetic unfocused ultrasound treatment;

automatically adjusting by a control circuitry connected to said memory said stored values of said at least one treatment parameter based on said stored one or more safety indications;

signaling at least one ultrasound transducer to emit unfocused ultrasound energy to said selected treatment target according to said automatically adjusted values.
Example 33. A method according to example 32, wherein said storing comprises storing indications related to location of at least one blood vessel and/or at least one nerve at a selected treatment target, and wherein said automatically adjusting comprises automatically adjusting said stored values of said at least one treatment parameter based on said stored location of said at least one blood vessel and/or said at least one nerve at said selected treatment target.
Example 34. A method according to any one of examples 32 or 33, wherein said storing comprises storing indications related to pain sensitivity of said specific subject, and wherein said automatically adjusting comprises automatically reducing ultrasound intensity in at least 5% and/or automatically increasing post cooling time duration in at least 5% base on said stored pain sensitivity indications.
Example 35. A system for delivery of a cosmetic unfocused ultrasound skin treatment, comprising:

an ultrasound applicator comprising one or more ultrasound transducers;

a camera configured to capture an image of an upper body of a subject and of said ultrasound applicator;

a control circuitry functionally connected to said camera, and configured to determine a position of said ultrasound applicator and/or said one or more transducers on said subject upper body according to signals received from said camera.
Example 36. A system according to example 35, wherein said camera is configured to capture an image of an upper body of a subject before, during and/or after movement of the subject, and wherein said control circuitry is configured to determine a position of said ultrasound applicator and/or said one or more transducers on said subject upper body according to signals received from said camera taking into consideration said subject movement.
Example 37. A system according to example 35, wherein said control circuitry is configured to generate a map of treated locations on said upper body based on said received camera signals; and wherein said system comprises a memory for storing said map.
Example 38. A system according to example 37, wherein said camera is configured to capture an image of an upper body of a subject before, during and/or after movement of the subject, and wherein said control circuitry is configured to generate said map taking into consideration said subject movement.
Example 39. A system according to any one of examples 35 to 38, wherein said camera is configured to move relative to said upper body of said subject.
Example 40. A system according to any one of examples 35 to 39, wherein said upper body comprises facial and/or neck body regions.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof.

Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data.

Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a block diagram of a system for applying ultrasound to tissue, according to some embodiments;

FIG. 2 is a flowchart of applying ultrasound energy to tissue while controlling heating of the tissue surface, according to some embodiments;

FIG. 3 schematically illustrates activation of an array of ultrasound transducers at various frequencies, and a thermal effect on tissue surface being treated by the transducers, according to some embodiments;

FIG. 4 is an exemplary configuration of an ultrasound applicator comprising a cooling module, according to some embodiments;

FIGS. 5A-B are exemplary graphs of activation of an array of ultrasound transducers, according to some embodiments;

FIG. 6 is a flowchart of a method for cosmetic ultrasound skin treatment, according to some embodiments;

FIG. 7 is a schematic diagram of a system for ultrasound skin treatment, according to some embodiments;

FIG. 8 is a schematic illustration of a system, according to some embodiments of the invention;

FIG. 9A is a flow chart of a process for treating skin tissue using ultrasound, according to some embodiments of the invention;

FIG. 9B is a flow chart of a process for personalizing a skin tissue treatment, according to some embodiments of the invention;

FIG. 9C is a flow chart of a process for modifying a skin tissue treatment to increase treatment safety, according to some embodiments of the invention;

FIG. 9D is a flow chart of a process for controlling application of ultrasound energy according to position of the ultrasound applicator and/or transducers, according to some embodiments of the invention;

FIG. 9E is a schematic diagram showing information flow between the ultrasound system, a remote device, a user of the system and a treated subject, according to some embodiments of the invention;

FIG. 10 is an image showing different facial and neck regions, according to some embodiments of the invention;

FIGS. 11A-L and 12A-H are photographs of facial and/or neck regions before and after a treatment performed as part of a clinical study and in accordance with some embodiments of the invention; and

FIGS. 12I-L are images and 3D models of facial regions before (FIGS. 12I-12J) and after (FIGS. 12K and 12L) a treatment performed as part of a clinical study and in accordance with some embodiments of the invention.

### DETAILED DESCRIPTION OF SPECIFIC EMBODIMENTS OF THE INVENTION

The present invention, in some embodiments thereof, relates to treating tissue using ultrasound energy and, more particularly, but not exclusively, to an ultrasonic transducer and applicator for skin treatments.

A broad aspect of some embodiments relates to treating tissue using unfocused ultrasound, for example non-converging ultrasound, while optionally cooling at least a portion of the tissue to reduce thermal damage to the tissue surface and/or other tissue layers. Some embodiments relate to controlled heating of tissue. Optionally, heat is applied to the tissue by an applicator comprising a plurality of transducers configured to emit unfocused ultrasound energy, the applicator comprising a cooling module configured to cool at least a surface of the tissue (e.g. skin) by applying cooling via the transducers that come in contact with the skin. In some embodiments, the extent of heating and/or cooling are controlled to obtain thermal damage at a selected depth relative to the tissue surface. In some embodiments, a structure and/or size (e.g. thickness) and/or materials of the transducer are selected to optimize heat transfer, such as heat transfer from the emitting surface of the transducer to the tissue and/or heat transfer from a cooling module of the applicator, via the transducer, to the tissue.

According to some embodiments, the treatment described in this application is a cosmetic treatment that cause no therapeutic effect. In some embodiments, selection of subjects for the treatment is performed so there is no therapeutic effect.

An aspect of some embodiments relates to an applicator configured to apply unfocused ultrasound to a tissue volume while maintaining the tissue surface cool enough to reduce or prevent thermal damage to the tissue surface. In some embodiments, the applicator comprises an array of ultrasonic transducers arranged side by side on a ribbed frame with thermal isolation between the transducers.

Optionally, thermal isolation is achieved by spacing the transducers apart such that air and/or other material isolates between them.

In some embodiments, the transducers and/or tissue surface contacting the applicator are actively cooled, for example by a thermo electric cooler (TEC) element used in conjunction with a heat exchanger, and/or by circulation of fluid such as water and/or antifreeze and/or by a gas. Additionally or alternatively, the transducers and/or tissue surface are passively cooled, for example by a thermal reservoir block (e.g. a cooled block of copper).

In some embodiments, cooling is applied to prevent over heating of the transducer. Additionally or alternatively, cooling is applied to reduce a temperature of the tissue surface. Optionally, cooling of the tissue surface is achieved via the applicator, for example by cooling the transducer's emitting surface to a temperature lower than a current temperature of the tissue surface. Cooling may be applied to the tissue before, during and/or after applying energy.

In some embodiments, the transducer is selected to be thin enough so as to provide for cooling of the tissue via the transducer. Optionally, using a thin ultrasound emitting element, e.g. a PZT plate having a thickness between 90-250 microns, allows for cooling of the tissue surface via the transducer, even when high intensity and/or high frequency ultrasound energy is emitted by the transducer. As the resonance frequency of the PZT plate is determined by a thickness of the plate, a potential advantage of using a thin plate may include the ability to use high frequencies, for example between 8-22 MHz. In some embodiments, cooling is applied to the tissue to control or limit energy dissipation inside the tissue.

In some embodiments, the applicator comprises one or more temperature sensors (e.g. thermistors and/or thermocouples) configured to indicate a temperature of the one or more transducers and/or to indicate a temperature of the tissue, e.g. of the tissue surface. Optionally, cooling is controlled in accordance with temperature feedback provided by the one or more temperature sensors. In some embodiments, one or more temperature sensors are configured to indicate a temperature of the frame carrying the transducers. In some embodiments, one or more temperature sensors are configured to indicate a temperature of a heat exchanger and/or other components of the applicator's cooling system.

An aspect of some embodiments relates to a flexible applicator for applying ultrasound energy to tissue, comprising one or more ultrasound emitting elements sandwiched between two layers of flexible film (e.g. Kapton). In some embodiments, the emitting elements are arranged side by side. In some embodiments, electrical circuitry configured for activating the emitting elements is embedded and/or printed on an inner side of one or both of the film layers, facing the emitting elements. Optionally, the circuitry comprises thermoresistors configured for indicating a temperature of the tissue and/or a temperature of the emitting elements.

In some embodiments, the applicator can be flexed to be positioned on non-flat tissue surfaces, such as on the forehead and/or around the neck. Optionally, each of the ultrasound emitting elements is narrow enough so as to reduce interference with bending, folding and/or otherwise shaping the flexible applicator. In some embodiments, the emitting elements are spaced enough from each other so that a flexible film portion in between them remains wide enough to be bent or otherwise flexed, enabling moving one element with respect to its adjacent element. An aspect of some embodiments relates to controlling a thermal effect on tissue at located different depths. In some embodiments, a first thermal effect is produced on tissue located at a first depth, and a second thermal different than the first thermal effect is produced on tissue located at a second depth, different from the first depth. Some embodiments relate to controlling a thermal effect on a tissue surface by exciting adjacent ultrasound transducers at different frequencies. In some embodiments, at least two transducers are excited, a first transducer at a frequency suitable for producing thermal damage to deeper tissue layers of the tissue and a second transducer at a frequency suitable for locally heating the tissue surface, the second frequency being at least 10% or at least 10% lower than the treating frequency of the first transducer.

In some embodiments, one or more transducers are excited at treatment frequencies (e.g. between 9-22 MHz), while one or more other transducers, for example transducers positioned in between the treating transducers, are excited at a frequency different than the treatment frequency (for example activated at a frequency which is twofold the resonance frequency) to produce sufficient heat for avoiding over-cooled tissue surface regions (overcooling may occur in tissue regions which are contacted by the actively cooled applicator, but are not contacted by the treating transducers). Alternatively, the non-treating transducers are not activated. Optionally, when not activated, the transducers are effective to cool the tissue surface in the vicinity of the heating transducers. In some cases, heating of the tissue surface is obtained by using relatively low power energy, optionally applied over a longer duration, for example as compared to high power which may result in undesired non-linear effects.

In some embodiments, the different frequencies are selected in accordance with ultrasound attenuation in the tissue. Optionally, increasing the frequency results in faster energy absorption in the tissue, such that the tissue layers closer to the emitting element are heated more than deeper tissue layers.

Additionally or alternatively to using different frequencies, the thermal effect is controlled by setting powering of the transducers, for example so that the second transducer (e.g. non-treating transducer)'s efficiency is relatively low, producing heating of the emitting element as a byproduct of activation which in turn heats the tissue surface. In some embodiments, heating (e.g. of the tissue surface) is provided by a heating element, for example a heating element mounted a tissue facing portion of the applicator.

An aspect of some embodiments relates to treating tissue by targeting a tissue layer and/or a tissue type located at a selected depth with respect to a surface of the tissue. Some embodiments relate to treating skin (e.g. to cause tightening of the skin) by producing controlled thermal damage at a depth of between 0.5-3 mm from the epidermis, using unfocused ultrasound energy.

In some embodiments, the unfocused ultrasound energy is applied to produce multiple spaced apart thermal damage lesions in the tissue, for example in the reticular dermis layer of the skin. In some embodiments, the lesions are substantially cylindrical. Alternatively, the lesions are of a different geometry, or optionally arbitrary shaped. A potential advantage of forming spaced-apart lesions may include that non-damaged tissue between lesions may promote healing by generating growth of elastin and/or collagen fibers. In some embodiments, use of unfocused ultrasound enables producing a repeatable spatial lesion pattern. A potential advantage of unfocused ultrasound may include covering a relatively wide surface area, reducing a need for repetitive movement of the applicator and potentially obtaining a more uniform distribution of the damage, for example as compared to use of focused ultrasound.

In some embodiments, a thermally damaged region extends between the spaced apart lesion and optionally connects the regions. For example, a thermally damaged layer of connective tissue (e.g. fat tissue) may extend between two or more cylindrical lesions produced in the reticular dermis of the skin, extending for example at the bottom of the lesions.

In some embodiments, the unfocused energy selectively targets fibrotic tissue (e.g. collagen fibers), while its effect on other types of tissue such as fat and/or connective tissue is relatively small since a sensitivity of these tissue types to the applied heat is reduced relative to the sensitivity of the fibrotic tissue, so that fat forms a natural barrier to the thermal damage.

In some embodiments, treatment parameters are selected for obtaining a desired effect. In an example of parameter selection, an intensity of the emitted ultrasound is selected to be between 8-40 W/cm\hat{ }2, between 12-22 W/cm\hat{ }2, between 10-17 W/cm\hat{ }2, between 14-18 W/cm\hat{ }2 to produce thermal damage in the dermis yet avoid damage to the epidermis; or, for example, above 22 W/cm\hat{ }2 to produce thermal damage in the dermis and in the epidermis, if such is desired. In some embodiments, selection of intensity should be correlated with the excitation duration and/or other parameters such as the extent of active cooling applied. In an example, intensities listed above are applied over a 4 second excitation duration, along with active cooling of the transducer base to a temperature of −10 degrees Celsius. Other examples of treatment parameters include a duration of treatment, a number of repetitions, excitation frequency, a number of activated transducers, and/or others. In some embodiments, treatment parameters are selected to obtain damage at a selected depth, such as between 0.5 to 5 mm from the epidermis. In some embodiments, treatment parameters are selected to obtain lesions of a specific size or geometry, for example lesions having a length of 1-4 mm.

Some embodiments relate to a system for aesthetic treatment, comprising an ultrasound applicator for example as described herein, a console and/or a user interface. In some embodiments, the applicator is configured to be moved across a surface of the skin (e.g. facial skin). In some embodiments, the system is configured to receive input, such as input pertaining to a desired effect, and to automatically select treatment parameters for obtaining that effect.

An aspect of some embodiments relates to obtaining a desired effect on the tissue (and optionally avoiding non-desired effects) by targeting a tissue layer to be heated. In some embodiments, a tissue layer is targeted and heated while other tissue layers and/or tissue located sideways (i.e. on a horizontal axis) remain substantially unaffected.

Desired effects include, for example, one or more of smoothing out wrinkles; reducing a visibility of stretch marks; evening out skin complexion and/or other effects.

In some embodiments, the effects comprise short term effects, long term effects, or a combination of both.

In some embodiments, treatment parameters are selected for producing a short term effect, such as one that is visible as soon as several minutes or several hours post treatment. In some embodiments, the treatment parameters are selected for producing a short term effect that lasts at least 6 hours, at least 1 day, at least 3 days, at least 1 week or intermediate longer or shorter time periods. Additionally or alternatively, the treatment parameters are selected for producing a long term effect, such as one that is visible only at 3 weeks post treatment, 2 months post treatment, 6 months post treatment or intermediate, longer or shorter time periods. In some embodiments, the treatment parameters are selected for producing a long term effect that lasts at least 1 month, at least 3 months, at least 1 year, at least 5 years or intermediate, longer or shorter time periods.

In some embodiments, a short term effect is obtained substantially without damaging a surface of the tissue. In some embodiments, a short term effect is obtained without a long term effect. In some embodiments, obtaining of short term effects is associated with a thermal damage sufficient to cause an inflammatory effect, including for example edema, irritation, swelling and/or others. Optionally, the thermal damage is limited only to an extent that results in inflammation but does not induce long term effects such as fibroblast penetration and/or substantial inducing of collagen and/or elastin generation.

In some embodiments, obtaining of long term effects is associated with a higher extent of thermal damage, such as one that induces generation of collagen and/or elastin and/or a general healing reaction. In some embodiments, a deeper tissue layer is targeted for obtaining a long term effect as compared to a layer that would be targeted for obtaining a short term effect.

In some embodiments, the system is configured to receive as input one or more of: a desired effect, a non-desired effect, a timing of the effect (e.g. short term or long term), a time period over which the effect should last, and/or other input, and to automatically select treatment parameters suitable for obtaining that effect. For example, the system selects parameters suitable for targeting a specific tissue layer while avoiding damage to other layers.

An aspect of some embodiments relates to combining ultrasonic treatment with one or more additional aesthetic treatments, such as filler injection treatment, topical cremes, neuro-toxin injection (BOTOX) and/or other treatments. In some embodiments, ultrasound treatment is applied as preparation for a second treatment. In some embodiments, ultrasound treatment affects the tissue in a manner that facilitates applying the second treatment. Additionally or alternatively, the two treatments work together for obtaining an effect, optionally an effect that cannot be obtained by each treatment separately. Optionally, applying both treatments obtains a desired effect in a time shorter than would have been required if each of the treatments was applied alone.

In an example, in the case of filler injection, ultrasound can be applied to cause loosening of connective tissue which may facilitate the process of injection; in another example, ultrasound is applied to thermally damage tissue at or adjacent a site of injection, for example to induce generation of a new collagen/elastin matrix.

Some embodiments relate to a method for obtaining an immediate visible effect on skin, comprising determining a time by which an effect should be visible; and applying heating to tissue underlying the epidermis, without thermally damaging the epidermis, less than 24 hours before the determined time. In some embodiments, unfocused ultrasound is applied by contacting said skin. In an exemplary application, a subject is treated in order to prepare for an event occurring on the same day or a day after. Optionally, the effect lasts over 1 day, 2 days, 5 days, 1 week or intermediate, longer or shorter time periods.

An aspect of some embodiments relates to delivery of a personalized ultrasound skin treatment. In some embodiments, the personalized treatment is a personalized non-converging ultrasound skin treatment, for example a cosmetic skin treatment. In some embodiments, the treatment is personalized, for example, in order to provide an efficient treatment of the skin, for example reducing the appearance of wrinkles and/or skin flattening, for a specific user in a short time period.

According to some embodiments, information about a subject selected for the treatment, is collected. In some embodiments, the subject specific information comprises clinical information, for example information related to the clinical condition of the subject, drug regime, medical history, and/or known pathologies for example skin-related pathologies. Optionally, the clinical information includes history of mental diseases. Optionally, the clinical information includes clinical information of family members.

According to some embodiments, information about a subject comprises information related to a potential treatment target of the skin. In some embodiments, treatment target information comprises tissue composition, for example thickness of a fat layer, depth of a treatment target volume, mechanical properties of the tissue, stretching capability of the skin, presence of wounds, scars or necrotic tissue in the potential treatment target. In some embodiments, treatment target information comprises information related to wrinkles in the treatment target, for example size, depth and/or density of wrinkles per skin surface area.

According to some embodiments, values of at least one treatment parameter are changed according to the collected information. In some embodiments, the treatment parameter comprises at least one of energy intensity, total amount of energy per surface area or region for a treatment session or overall treatment, duration of energy delivery, and/or number of pulses per region. Alternatively or additionally, the treatment parameter comprises number of treatment sessions needed to reach a desired result, overall treatment time needed to reach a desired result.

According to some embodiments, the treatment is personalized for a specific subject during the overall treatment, for example between and/or during treatment sessions, optionally by evaluation of the treatment results and modification of the at least one treatment parameter if needed.

An aspect of some embodiments relates to delivery of a safe ultrasound treatment, for example a cosmetic ultrasound treatment to a specific subject. In some embodiments, specific values of at least one treatment parameter, for example energy intensity and/or duration of energy delivery, are selected in order to provide the safe ultrasound treatment. In some embodiments, a safe ultrasound treatment is a treatment that does not result with a nerve injury and/or paralysis of one or more muscles, for example facial muscles, for a time period longer than 6 hours, for example a time period longer than 7 hours, 10 hours, 12 hours or any intermediate, shorter or longer time period from an end of ultrasound energy delivery. Additionally or alternatively, a safe ultrasound treatment is a treatment that does not result with pain sensation, for example pain sensation in the treated area, for a time period longer than 30 minutes, for example a time period longer than 40 minutes, longer than 90 minutes or any intermediate, shorter or longer time period, from an end of ultrasound energy delivery.

According to some embodiments, information from a subject is collected, for example to determine a location of nerves and/or blood vessels in or near a selected treatment target. Alternatively or additionally, the information collected from the subject is used to estimate pain level sensitivity of the subject, for example at the selected treatment target. Optionally, the pain level sensitivity of the subject comprises sensitivity to pain caused by excess of heat or cold.

According to some embodiments, one or more nerves located at a depth in a range of 0.5 mm-3 mm, for example 0.5 mm-2 mm, 1 mm-2.5 mm, 2 mm-3 mm or any intermediate, smaller or larger range of depth from the skin surface, are identified in skin regions, for example potential treatment regions.

According to some embodiments, specific values of the at least one treatment parameter are selected according to a distance of the ultrasound transducers from a determined location of identified blood vessels and/or nerves, and/or according to a depth of the identified nerves and/or blood vessels from the skin surface. Additionally or alternatively, specific values of the at least one treatment parameter are selected according to the estimated pain level of a subject in the treated area. In some embodiments, the energy delivery intensity is adjusted to be at least 5% lower, for example at least 10% lower, at least 20% lower or any intermediate, smaller or larger percentage value of the ultrasound energy intensity level that causes intolerable pain sensation in a specific subject.

According to some exemplary embodiments, a link between the number of Joules per pulse, and the energy intensity in W/cm² depends on one or more of, number of transducers used and the time duration in which a specific intensity is emitted, for example pulse duration, pre-pulse cooling duration, post-pulse cooling duration, and ultrasonic frequency.

According to some exemplary embodiments, the ultrasonic energy is delivered in one or more pulses of unfocused ultrasonic energy. In some embodiments, the pulses are delivered through a surface area size in a range of 3 mm²-7 mm² or any intermediate, smaller or larger range of values. In some embodiments, each pulse has an intensity in a range of 5 W/cm²-60 W/cm², for example 10 W/cm²-30 W/cm² 10 W/cm²-17 W/cm², 15 W/cm²-25 W/cm², 20 W/cm²-30 W/cm², 25 W/cm²-50 W/cm², 40 W/cm²-60 W/cm² or any intermediate, smaller or larger range of values. In some embodiments, a time duration in which said ultrasonic energy is actively transmitted is in a range of 1-10 seconds per pulse, for example 1-5 seconds, 3-7 seconds, 6-10 seconds or any intermediate, smaller or larger range of values. In some embodiments, the unfocused ultrasonic energy is delivered to cover one or more skin regions having a surface area size in a range of 2 cm²-150 cm², for example 2 cm²-50 cm², 10 cm²-100 cm², 50 cm²-150 cm² or any intermediate, smaller or larger range of values, when optionally delivered from a fixed position. In some embodiments, the unfocused ultrasound energy is delivered by 1-20 transducers, for example a single transducers, 1-10 transducers, 5-15 transducers, 10-20 transducers or any intermediate, smaller or larger number of ultrasound transducers.

According to some exemplary embodiments, the ultrasonic energy is delivered with treatment parameter values adjusted to generate a high thermal effect of about 55-65° C. in tissue layers in a depth of about 0.5-6 mm, for example about 0.7-1.2 mm, about 0.9-1.5 mm, about 1-2 mm, about 2-4 mm, about 3-6 mm, about 2-6 mm or any intermediate, smaller or larger range of values, from the external surface of the skin. In some embodiments, the ultrasonic energy is delivered with treatment parameter values adjusted to generate a moderate thermal effect of about 47-55° C. in tissue layers in a depth of about 0.4-0.7 mm and about 2-2.5 mm from the external surface of the skin.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

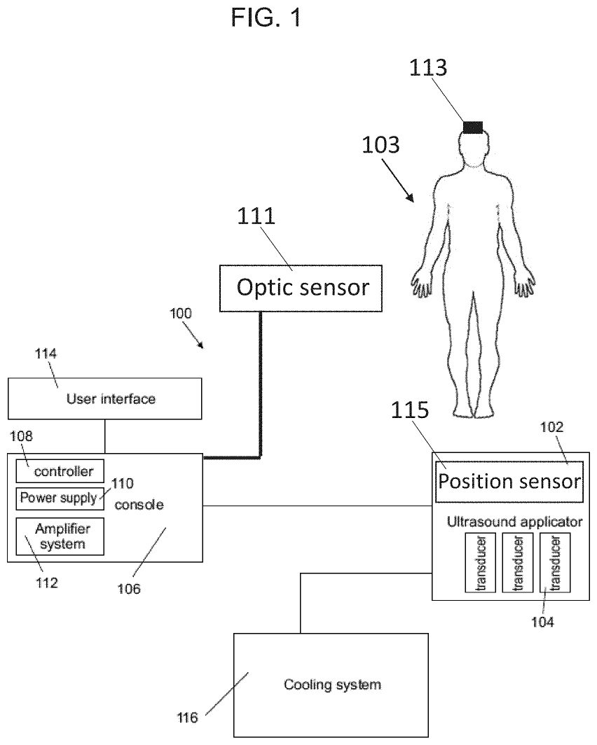

Referring now to the drawings, FIG. 1 is a block diagram of a system for applying ultrasound to tissue, according to some embodiments.

In some embodiments, system 100 comprises an ultrasound applicator 102, configured for applying ultrasound energy to tissue, such as skin. In some embodiments, applicator 102 comprises a handle operable by a clinician, such as a physician or a nurse.

In some embodiments, applicator 102 comprises one or more ultrasound emitting elements, such as one or more ultrasound transducers 104. In an example, applicator 102 comprises an array of ultrasound transducers, for example comprising 5 transducers, 7 transducers, 9 transducers, 12 transducers or intermediate, larger or smaller number of transducers.

In some embodiments, system 100 comprise a console 106. In some embodiments, console 106 comprises a controller 108. In some embodiments, controller 108 is configured to control operation of the system, for example controlling emission of ultrasound energy by applicator 102. In some embodiments, the controller comprises a memory which stores, for example, setup data, records of previous treatments, and/or others. In some embodiments,

In some embodiments, console 106 comprises one or more components for operating the system, for example including power supply 110 (and/or connection to an external power source), an amplifier system 112 and/or other components such as an oscilloscope. In some embodiments, console 106 is portable, for example placed on a cart. In some embodiments, console 106 comprises a user input module. Optionally, a user (e.g. physician) inserts one or more of: treatment parameters, patient data, desired and/or non desired treatment effects, and the controller selects one or more of: treatment parameters, a tissue layer to be targeted, a number of treatments, timing of treatments and/or treatment duration according to the inserted input.

In some embodiments, system 100 comprises a user interface 114 for receiving input from a user such as a physician and/or for providing information to the user. In some embodiments, user interface 114 is configured for receiving operation parameters, for example including energy parameters such as frequency, intensity, and/or usage parameters such as treatment duration. In some embodiments, user interface 114 is configured to receive patient data (e.g. age, weight, height, gender, medical condition, and/or other patient related data). Optionally, user interface 114 is configured to automatically select a treatment regimen in accordance with the patient parameters.

In some embodiments, user interface 114 comprises a display. In some embodiments, user interface comprises a computer such as a laptop or a tablet computer.

In some embodiments, system 100 comprises a cooling system 116. In some embodiments, cooling system 116 is configured to cool one or more portions of applicator 102, for example configured for cooling transducers 104. Additionally or alternatively, cooling system 116 is configured to cool a tissue surface to which the energy is applied, for example cooling tissue being contacted by the applicator and/or surrounding tissue.

In some embodiments, cooling system 116 comprises a circulating coolant in the form of liquid and/or gas, for example water or antifreeze fluid. Optionally, circulation is actuated by a pump.

In some embodiments, cooling system 116 comprises an active cooling element, such as a thermoelectric cooler. In some embodiments, cooling system 116 comprises a fan. In some embodiments, a chiller is used for cooling the liquid and/or gas.

Additionally or alternatively, cooling system 116 comprises a passive cooling element, such as a thermal reservoir block, for example a copper block. Optionally, the copper block is precooled to a temperature sufficient to provide cooling of the transducers and/or the tissue surface throughout the treatment.

In some embodiments, cooling system 116 is coupled to applicator 102. In some embodiments, cooling system 116 is an inherent component of applicator 102.

In some embodiments, system 100 is activated to emit ultrasound energy towards the treated tissue. In some embodiments, the ultrasound energy is non-focused, for example non-converging, energy. Alternatively, in some embodiments, the ultrasound energy is focused. In some embodiments, parameters of the emitted energy are selected to produce a thermal damage effect in the treated tissue. In some embodiments, the parameters are selected to achieve a certain extent of damage (e.g. dimensions of the damaged tissue volume) and/or a certain level of damage (e.g. minor damage, intermediate damage, strong damage) and/or a certain location of damage.

In some embodiments, transducers 104 are cooled by cooling system 116 to control a thermal effect on the tissue, for example to reduce thermal damage (e.g. necrosis, protein denaturation, and/or blood thrombosis) to the tissue surface.

In some embodiments, a thickness of a transducer 104 is selected to be low enough so that the transducer is efficiently cooled by the cooling system. In some embodiments, the transducer cools the tissue surface it comes in contact with.

A potential advantage of a thin transducer may include improved resistance to breakage, for example breakage caused by thermal stresses resulting from strong cooling applied before, after and/or during excitation of the transducer.

In some embodiments, each transducer is configured to be excited independently of the other transducers. In some embodiments, amplifier system 112 comprises a separate power amplifier for each of the transducers.

Alternatively, two or more transducers are configured to be excited together.

Optionally, the transducers are connected in a parallel configuration.

Alternatively, the transducers are connected in a serial combination.

Alternatively, the transducers are connected in a combination of serial and parallel sets.

In some embodiments, the circuitry comprises one or more electrical components (e.g. resistors, coils and/or capacitors) for controlling powering of each of the transducers, for example by setting an impedance on a branch leading to one of the transducers.

In some embodiments, the applicator is wirelessly activated. Optionally, the applicator is battery powered. In some embodiments, the battery is charged via a charging station and/or by wireless induction (e.g. using electromagnetic radiation).

In some embodiments, the applicator is portable.

According to some exemplary embodiments, system 100 comprises at least one optic sensor 111, for example a camera. In some embodiments, the optic sensor is connectable, for example functionally connectable to the console 106 body. Alternatively, the optic sensor 111 is an integral part of the console. In some embodiments, the optic sensor is movable, and is configured to image an upper body of a subject from different angles and/or positions relative to the upper body of the subject. Optionally, the optic sensor is mounted on a movable arm mechanically connected to the console or to a bed or a chair on which said subject is positioned.

In some embodiments, the optic sensor 111 is electrically connected via wires or wirelessly to the controller 108. In some embodiments, the optic sensor 111 is configured to image, for example to take video or stills images of the subject 103, for example images of a treatment region on the body of the subject 103, for example on the upper body of the subject. In some embodiments, the upper body of the subject comprises the face and/or neck of the subject.

According to some exemplary embodiments, the applicator 102 comprises at least one position sensor, for example position sensor 115, configured to provide information regarding the position of the applicator and/or position of one or more of the transducers 104. Optionally, the position sensor is a marker that can be visualized by the optic sensor 111. In some embodiments, the system 100 comprises a marker 113 or at least one position sensor attached to the subject 103.

According to some exemplary embodiments, the ultrasound system, for example the controller 108 determines a position of the ultrasound applicator 102 and/or a position of one or more ultrasound transducers 104 based on signals received from the optic sensor 111, during the delivery of the ultrasound treatment. Optionally, the controller determines the position of the ultrasound applicator 102 and/or a position of one or more ultrasound transducers 104 based on signals received from the optic sensor 111, and a registered position of the marker 113 and/or using signals received from the position sensor 115 of the applicator 102.

According to some exemplary embodiments, based on a known position of the applicator 102 and/or transducers 104, the system 100 generates an indication, for example a human detectable indication to a user to continue delivery of ultrasound energy in a specific location, for example a specific facial or neck location. Alternatively or additionally, the system 100, for example controller 108, is configured to generate a map of the treated regions, based on the determined position of the applicator and/or transducers. Optionally, based on the determined position, the system 100 generates an alert signal if the ultrasound applicator 102 and/or transducers 104 move to an undesired position, for example a position that is close to blood vessels and/or nerves that can be affected by the treatment.

According to some exemplary embodiments, the system 100, for example the controller 108 is configured to generate a map of the subject, for example a map of an upper body of the subject. In some embodiments, the map is generated based on signals received from the optic sensor 111, for example from the camera, and/or based on signals of the position sensor 115.

According to some exemplary embodiments, the optic sensor 111, for example the camera, is configured to image a subject body while the subject body moves, for example before, during and/or after ultrasound energy delivery. In some embodiments, the generated map takes into consideration the movement of the subject, for example by comparing images of the subject to a 3D model of the subject. Optionally, the 3D model is generated using optic sensor 111 and/or an additional camera, for example an external camera. In some embodiments, the system 100, for example, the controller 108 of the system is configured to determine a position of the applicator relative to the body of the subject, while taking into account subject movement, for example using the 3D model of the body.

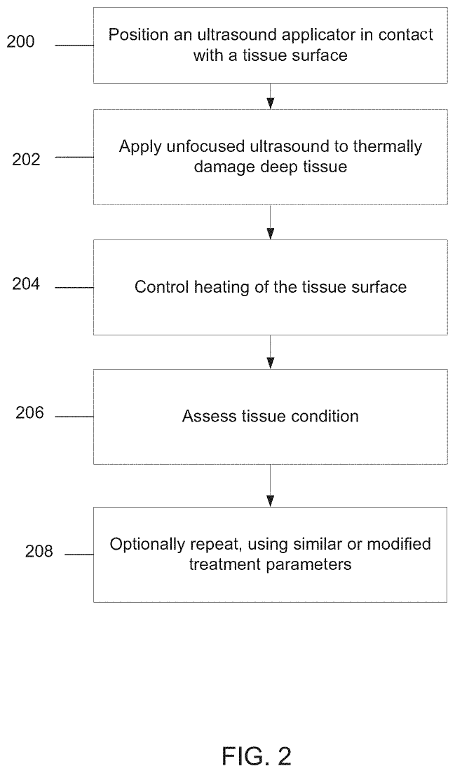

FIG. 2 is a flowchart of applying ultrasound energy to tissue while controlling heating of the tissue surface, according to some embodiments.

In some embodiments, an ultrasound applicator comprising one or more ultrasound transducers is positioned in contact with a tissue surface (200), for example in contact with the skin. In some embodiments, contact is between an external surface of the one or more ultrasound transducers and the tissue. Optionally, contact is achieved when at least 60%, at least 80%, at least 90% or intermediate, higher or smaller percentage of an external surface area of the ultrasound transducer(s) contacts the tissue surface.

In some embodiments, unfocused ultrasound energy is applied to thermally damage deep target tissue (202), for example tissue located at least 1.5 mm, at least 3 mm, at least 5 mm or intermediate, shorter or longer distances beyond the tissue surface.

In some embodiments, unfocused ultrasound energy is applied for treating skin tissue. Optionally, the energy is applied selectively, for example to cause thermal damage to deeper skin layers such as the dermis while damage to upper layers such as the epidermis is reduced or prevented. In an example, dermis tissue at a depth of 2 to 1.55 mm is thermally damaged.

In some embodiments, the applied energy is sufficient to raise a temperature of the target tissue, for example to a temperature between 50-80 degrees Celsius, such as 60-70 degrees Celsius, 55-65 degrees Celsius, 70-75 degrees Celsius or intermediate, higher or lower temperatures.

Optionally, the energy is applied over a time period between 1-60 seconds, such as 5-10 seconds, 10-20 seconds, 15-30 seconds or intermediate, longer or shorter time periods.

In some embodiments, the applied energy ablates the target tissue. In some embodiments, target tissue (e.g. collagen) is denatured and/or coagulated.

In some embodiments, the energy is applied selectively to thermally damage lesions separated by areas area of healthy, substantially undamaged tissue. A potential advantage of controlling application of the ultrasound energy to produce lesions separated by healthy tissue may include promoting healing, for example by inducing growth of elastin and/or collagen fibers.

In some embodiments, a lateral distance between thermally damaged lesions is between 1-5 mm, for example 2-4 mm, 3-5 mm, 1-2 mm or intermediate, longer or shorter distances. In some embodiments, each thermally damaged lesion is between 0.5 mm³-5 mm³ in volume, for example 2 mm³, 4 mm³, 1 mm³ or intermediate, larger or smaller volumes.

In some embodiments, the produced lesion is substantially cylindrical.

Alternatively, a lesion is spherical, cubical, cone shaped.

In some embodiments, the energy selectively targets tissue, for example targeting tissue of the reticular dermis such as collagen, elastic fibers and/or extrafibrillar matrix. Optionally, the effect of the emitted energy on other types of tissue such as fat tissue and/or connective tissue is small, so that fat tissue forms a natural barrier to the damage (e.g. a layer of fat tissue below the dermis). A potential advantage of applying unfocused ultrasound energy which produces a thermal effect that is naturally reduced or limited by certain types of tissue such as fat tissue may include reduced sensitivity to anatomical variations (e.g. inter-patient variations in tissue structure and/or thickness).

In some embodiments, heating of the tissue surface is controlled (204). In some embodiments, cooling is applied to the tissue surface to reduce a thermal effect of the emitted ultrasound beam on the tissue surface.

In some embodiments cooling is applied via the ultrasound transducer(s), for example by cooling a transducer's emitting surface to a temperature lower than a current temperature of the tissue. Additionally or alternatively, cooling is applied by delivering cold liquid and/or gas to the tissue, for example through designated holes formed in the transducer surface.

In some embodiments, heat is conducted to and/or from the tissue surface contacting the emitting surface of the ultrasound transducer.

In some embodiments, the transducer's emitting surface contacts the tissue directly. Alternatively, a thin layer of an isolating material such as Kapton and/or other polyimide film and/or Parylene and/or PEEK and/or PTFE and/or Silicon rubber and/or Latex, is disposed on the emitting surface of the transducer which faces the tissue. Optionally, a face of the applicator which faces the tissue is coated by a protective layer, for example a thin thermally and/or electrically insulating layer.

In some embodiments, cooling systems and/or methods for example as described herein are used for cooling the transducer's emitting surface (e.g. cooling using fluid circulation, a thermal reservoir block, a thermos electric cooler and/or other methods). Optionally, cooling is applied before, after and/or in between energy emission periods.

Additionally or alternatively, cooling is applied to tissue surface in a vicinity of the tissue area contacting the transducer's emitting surface. Optionally, non-active transducers and/or transducers activated using different parameters than the treatment parameters cool the tissue in a vicinity of the treating transducer.

Additionally or alternatively, cooling of the tissue surface before, during and/or after energy emission is achieved by directly cooling the tissue surface, for example using gel. In some embodiments, the gel is an ultrasonic conductive gel. Optionally, the gel fills up gaps between the transducer's emitting surface (or a coating thereon) and the tissue. Additionally or alternatively, a liquid filled balloon is used for cooling the tissue surface.

In some embodiments, one or more parameters are taken into consideration when controlling heating of the tissue surface, such as: heat dissipation to the surroundings (depending, amongst other parameters, on the ambient temperature); parameters of the emitted ultrasound beam (e.g. intensity profile, frequency profile, beam angle, beam shape,); the type of tissue being treated; thermal conductivity, thermal capacitance and/or heat dissipation coefficients of the tissue being treated; absorption and/or attenuation coefficients of the ultrasound waves in the tissue; a geometry and/or dimensions of the piezo element and/or other parameters.

In some embodiments, heating of the tissue surface is controlled by selecting a piezo element having a certain thickness and/or width. For example, the piezo element is selected with a thickness which defines a resonance frequency between 9-22 MHz.

In another example, a width of the piezo element is selected to provide an ultrasonic beam having a pre-defined opening angle, for example a width between 0.5-3 mm is selected to provide a beam having an opening angle between 5-45 degrees.

Optionally, increasing the beam angle (e.g. by providing a piezo element of increased width) reduces a thermal effect on the surface of the tissue.

In some embodiments, a temperature of the emitting surface of the transducer is determined. Optionally, the temperature is measured via one or more temperature sensors. Additionally or alternately, a temperature of the emitting surface is determined by measuring a capacitance of the transducer during excitation. In some embodiments, a temperature of a material coating the transducer's emitting surface (e.g. Kapton) is determined. Additionally or alternatively, tissue bio-impedance is measured (for example by stimulating the tissue via the transducers) as an indication of a thermal condition of the tissue. Additionally or alternatively, the tissue temperature is determined using ultrasound signals reflected from the tissue.

Optionally, the signals are received by the applicator (e.g. by one or more receivers configured on the applicator, and/or by one or more transceivers. In some embodiments, one or more transceivers are configured for both emitting the treating energy and receiving the reflected signals).

In some embodiments, a tissue condition is assessed (206). Optionally, the tissue condition is assessed during treatment and/or following treatment. In some embodiments, the extent of thermal damage is assessed.

In some embodiments, the extent of thermal damage is assessed by analyzing echo signals reflected from the tissue. In some embodiments, the device is configured to receive echo signals, and optionally the device's controller is configured for performing such analysis. Optionally, one or more of the applicator's transducers are configured to function as transceivers configured to receive the returning signals.

In some embodiments the tissue condition is assessed after a certain time period has passed from the treatment, for example 1 day, 1 week, 3 weeks, 1 month, 3 months or intermediate longer or shorter time periods from the treatment. For example, in some embodiments, a visible effect can be observed on treated skin following treatment, for example tightening of the skin.

Optionally, treatment is repeated (208). In some embodiments, treatment is repeated until a desired effect is achieved, for example visible tightening of the skin.

In some embodiments, one or more treatment parameters are modified, for example energy parameters (e.g. frequency, intensity); a temperature profile of the transducer(s); a treatment duration; a shape and/or size of the applicator.

In some embodiments, treatment is applied to tissue other than skin, for example tissue of the reproductive system, urinary tract, gastrointestinal tract, airways and/or any other tissue approachable via natural orifices of the body.

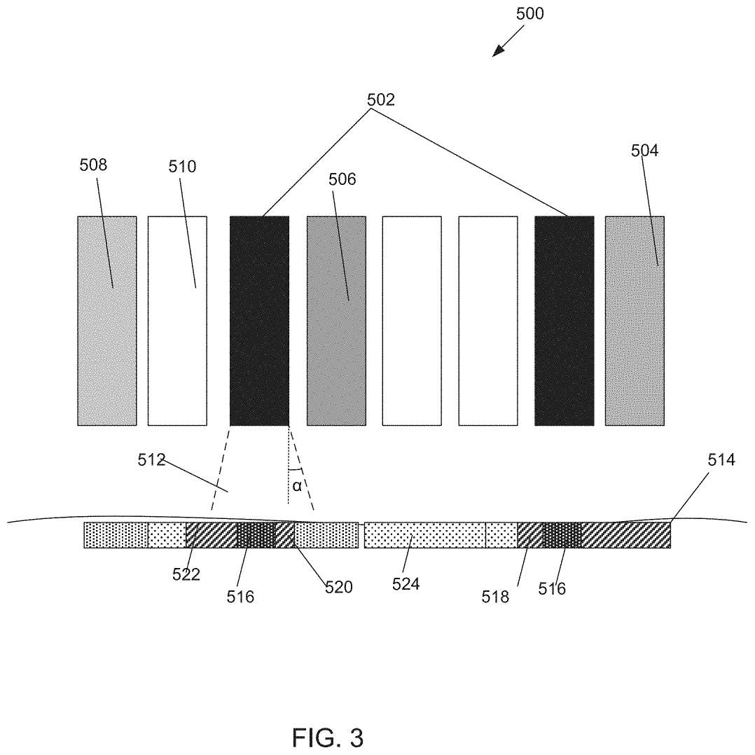

FIG. 3 schematically illustrates activation of an array 500 of ultrasound transducers at various frequencies, and a thermal effect on tissue surface being treated by the transducers, according to some embodiments.

In some embodiments, one or more transducers 502 are activated at a frequency suitable for thermally damaging deep tissue, for example a frequency between 8-22 MHz, 10-20 MHz, such as 11 MHz, 15 MHz, 18 MHz.

In some embodiments, one or more transducers 504, 506, 508 are activated at non-treating frequencies, for example at a frequency higher than 20 MHz such as 22 MHz, 33 MHz, 45 MHz, or at a frequency lower than 10 MHz, such as 9 MHz, 5 MHz, 2 MHz. Optionally, transducers 504, 506, 508 are activated at similar frequencies; alternatively, transducers 504, 506 508 are activated at various frequencies.

In some embodiments, one or more transducers 510 are not activated.

In some embodiments, energy emitted by the one or more transducers comprise unfocused ultrasound energy. Additionally or alternatively, energy emitted by the one or more transducers comprises focused ultrasound energy.

In some embodiments, unfocused ultrasound and focused ultrasound are applied simultaneously or successively. A potential advantage of applying focused ultrasound and unfocused ultrasound simultaneously and/or successively may include obtaining a stronger thermal effect on the tissue, as the unfocused ultrasound will heat tissue surrounding the focal point of the focused ultrasound, raising the temperature of the tissue at the focal point. Another potential advantage of using focused ultrasound may include accurately targeting individual treatment points that are isolated from each other.

In some embodiments, focused ultrasound generates a cavitation bubble cloud, for example when applied at a frequency between 0.25-0.2 MHz. Optionally, applying a non-focused ultrasound beam simultaneously or successively to the focused ultrasound heats the cloud region and may provide for targeted ablation of the cloud region. An exemplary embodiment in which focused and unfocused ultrasound energy may be applied together includes hair removal applications, in which the focused ultrasound generates cavitation inside the hair duct, and the unfocused beam, optionally applied at a higher frequency than the focused beam, intensifies heating of the hair duct. Other applications may include sweat gland treatments, acne treatments and/or other treatments.

In some embodiments, energy fields produced by adjacent transducers overlap.

Optionally, the adjacent transducers emit different energy types (e.g. focused ultrasound, unfocused ultrasound, RF). Optionally, the adjacent transducers are driven at different frequencies.

In some embodiments, overlapping fields generate a complex field which may include localized peaks of higher intensity.

In some embodiments, beam 512 comprises a substantially trapezoidal profile.

Alternatively, the beam comprises a different profile, such as a conical, rectangular, and/or other profiles. In some embodiments, an opening angle a of beam 512 is between, for example, 5-20 degrees, such as 10 degrees, 15 degrees, 19 degrees.

In some embodiments, an energy distribution of the emitted beam is controlled by selecting energy parameters. For example, in some embodiments, energy parameters (e.g. frequency and/or intensity) are selected so that the ultrasound field of the produced beam is stronger at base of the beam, for example effective to heat the contact point with the tissue more than at other beam portions. Additionally or alternatively, an intermediate portion of the beam is stronger, for example effective to heat tissue at a shallow depth from the surface. Additionally or alternately, a distant portion of the beam is stronger, for example effective to heat deep tissue regions.

FIG. 3 further shows a thermal effect of an array for example as described herein on the tissue surface 514. In some embodiments, tissue surfaces 516 effected (e.g. by being contacted by) treating transducers 502 are heated the most, for example heated to a temperature between 20-40 degrees Celsius. In some embodiments, tissue surfaces such as 518, 520 and/or 522 which are effected by both the treating transducer and the adjacent optionally cooler transducer are heated to a lower temperature, for example a temperature between 10-30 degrees Celsius. In some embodiments, tissue surfaces such as 524 located further away from the treating transducer are heated to a lower temperature, for example between 5-25 degrees Celsius.

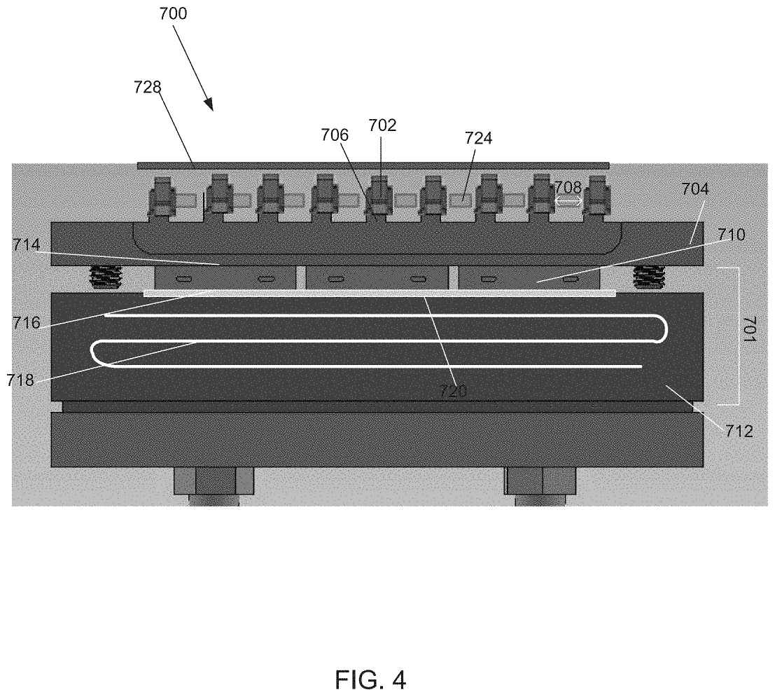

FIG. 4 is an exemplary configuration of an ultrasound applicator comprising a cooling module, according to some embodiments.

In some embodiments, applicator 700 comprises a one or more ultrasound transducers 702 (e.g. 9 transducers, 5 transducers, 15 transducers or intermediate, larger or smaller amount). In some embodiments, transducers 702 are mounted on a base 704. Optionally, each transducer 702 is mounted on a distally extending branch 706 of base 704. Optionally, transducers 702 are attached to base 704 by a thin layer of glue, for example thermally conductive and/or electrically conductive glue.

Optionally, for example as shown herein, branches are distanced away from each other a lateral distance 708 of between, for example, 1-6 mm, 2-4 mm, 0.5-3 mm or intermediate, shorter or longer distances. Optionally, a distance between the branches and/or a spatial orientation of the branches with respect to each other is selected in accordance with a lesion pattern intended to be formed in the treated tissue.

In some embodiments, a thermal and/or electrical isolation is configured in between branches 706. Optionally, air is allowed into the gaps defined between the branches for thermally and electrically separating between adjacent transducers.

Additionally or alternatively, a thermally and/or electrically isolating material such as polyurethane foam is disposed between the branches (not shown herein).

Alternatively, in some embodiments, base 704 including branches 706 is coated by a thermally and/or electrically isolating material (e.g. polyimide and/or Parylene, for example having a thickness between 10-20 microns). Optionally, the coating traps air in the gaps between the branches. Alternatively, in some embodiments, base 704 does not comprise branches, and the transducers are mounted directly onto the base. Optionally, base 704 is coated, at least in part, by a thermally and/or electrically isolating material e.g. polyimide and/or Parylene, for example having a thickness between 10-20 microns). In some embodiments, the coating comprises electrical circuitry (e.g. printed circuitry) configured for activating transducers 702 and/or for heating the tissue surface being contacted by the applicator, for example by heating the tissue directly and/or by heating the transducers.

In some embodiments, applicator 700 comprises a cooling module 701, configured for absorbing and/or dissipating heat away from the transducers and/or for actively and/or passively cooling the transducers. In some embodiments, cooling module 701 is configured to transfer heat away from the transducers at a rate fast enough to prevent over-heating of the transducers, such as overheating of an ultrasound emitting surface of the transducer. In some embodiments, the cooling rate is high enough to cool the transducers to a temperature lower than a current temperature of the tissue surface. Optionally, active cooling is provided. A potential advantage of cooling the transducers to a temperature lower than a current temperature of a surface of the treated tissue may include reducing the need for additional cooling elements, such as cooling elements configured to cool the tissue surface directly, as cooling is provided to the transducer and also via the transducer (e.g. via the transducer's emitting surface) cooling the tissue surface being contacted by the transducer. In some embodiments, cooling module 701 is controlled in accordance with activation of the transducers. Optionally, the cooling rate is high enough to overcome heating generated by the energy emitting transducers. Exemplary cooling rates may include 1 K/min or 5 K/min or 10 K/min, or 60 K/min or intermediate values, and heat transfer of 1-7 W/(m\hat{ }2K), or intermediate values.

In some embodiments, cooling module 701 comprises one more cooling elements, such as a Peltier element, for example in the form of a thermoelectric cooler (TEC) 710. Optionally, one or more TEC elements (e.g. 3 as shown herein) are positioned in contact with base 704. In some embodiments, base 704 comprises aluminum and/or copper and/or brass and/or stainless steel.

In some embodiments, the cooling module 701 comprises a heat sink 712.

Optionally, heat sink 712 is configured to absorb and/or dissipate heat away from TEC elements 710, for example disposed under the TEC elements.

In some embodiments, TEC element 710 is positioned in between base 704 and heat sink 712. Optionally, a distally facing surface 714 of the TEC contacts, at least in part, base 704; a proximally facing surface 716 of the TEC contacts, at least in part, heat sink 712. Optionally, distally facing surface 714 is the cooled side of the TEC; proximally facing surface 716 is the hot side of the TEC. Optionally, power supply to TEC is provided via a power lead (not shown herein).

In some embodiments, each transducer is coupled to a single TEC element.

Optionally, a substrate (e.g. ceramic substrate) configured on distally facing surface 714 is removed, and a direct coupling is produced between the electrical circuitry of the TEC element and the transducer. Such direct coupling may be advantageous, for example, for independently controlling cooling of each of the transducers, for example in an operation mode in which one or more transducers are activated with a first set of energy parameters (e.g. frequency, intensity) and one or more other transducers are activated with a second set of energy parameters.

In some embodiments, a heat transferring layer 720 is disposed between TEC 710 and heat sink 712, and/or between the distally facing TEC surface 714 and base 704, for example comprising a thermally conductive glue, paste and/or pad.

In some embodiments, heat sink 712 comprises a coolant 718, for example comprising fluid and/or gas and/or antifreeze. In an example, the coolant comprises water. Optionally, the coolant is circulated within the heat sink, for example using a pump (not shown herein). In some embodiments, the coolant is cooled by a chiller (not shown herein), for example disposed within heat sink 712 and/or disposed externally to applicator 700.

In some embodiments, cooling module 701 comprises a fan (not shown herein), configured for providing additional heat removal and/or for replacing one or more cooling elements as described hereinabove (e.g. a TEC and/or a heat sink).

Additionally or alternatively, cooling module 701 comprises a thermal reservoir block (not shown herein), for example a block of copper. Optionally, the thermal reservoir block is pre-cooled to a temperature sufficient to cool transducers 702, for example via base 704, enough to reduce or prevent thermal damage to a surface of the tissue.

In some embodiments, base 704 is mounted directly on heat sink 712.

Optionally, in such configuration, a temperature of coolant 718 is reduced to an even lower degree (e.g. as compared to an applicator in which a TEC element is used).

In some embodiments, a continuous PZT plate may be used, for example replacing the branched structure of base 704. In some embodiments, the continuous PZT plate is processed to define multiple, optionally independently operable emitting elements for example as described hereinbelow.

Additionally or alternatively, a continuous porous PZT plate is used. In some embodiments, the porous PZT plate is coated by an electrically conductive layer (e.g. a silver layer), and the layer is removed (e.g. etched) in a pattern suitable to produce separate electrodes for exciting the respective PZT portions contacting the electrodes.

Additionally or alternatively, multiple emitting elements are produced by placing separate electrodes on the top and/or bottom faces of the porous PZT plate. In some embodiments, when using a porous PZT plate, a thickness of the plate is selected to be lower than, for example, a thickness of a non-porous PZT element, since the speed of sound is lower in the porous material.

In some embodiments, applicator 700 comprises an arrangement including more than one base which carries transducers. In an example, two bases are arranged to oppose each other (e.g. defining a mirrored symmetry). Optionally, a distance and/or angular position between the bases is selected and/or modified to produce a specific lesion pattern in the treated tissue. Optionally, each of the bases is coupled to a separate TEC element.

In some embodiments, applicator 700 comprises one or more temperature sensors 724. In some embodiments, sensors 724 are placed in between transducers 702. Optionally, sensors 724 are coupled to a coating (e.g. a polyimide and/or parlyene coating of the base, for example as described hereinabove) and/or coupled to an isolating material configured between the branches. In some embodiments, sensor 724 is configured to indicate a temperature of transducer 702, for example a temperature of the emitting surface of the transducer. Additionally or alternatively, senor 724 is configured to indicate a temperature of the tissue surface. Optionally, temperature sensor 724 is positioned in proximity to the transducer, for example between 0.1mm to 1 mm from the transducer's emitting surface.

Additionally or alternatively, a temperature of the transducer is assessed by analyzing echo signals reflected by the tissue and received by applicator 700.

Optionally, applicator 700 comprises one or more ultrasound receiving elements. Optionally, one or more transducers 700 are configured to function both as emitter and as receivers.

In some embodiments, applicator 700 comprises one or more RF electrodes (not shown). Optionally, the RF electrodes are coupled to a coating of the base and/or coupled to an isolating material between the transducers. In some embodiments, the RF electrodes are used for applying additional heating to the tissue surface, for example so as to reduce thermal damage to the surface. Additionally or alternatively, the RF electrodes are used for measuring bio-impedance of the tissue. Optionally, bio-impedance measurements are performed to assess contact of the transducers with the tissue and/or as a measure of the tissue condition in response to treatment.

In some embodiments, a thin gel pad 728 (e.g. having a thickness between 0.1-1 mm) is disposed on a distal end of applicator 700. Optionally, gel pad 728 enhances contact between the transducers and the tissue. Optionally, gel pad 728 applies cooling to the tissue surface (e.g. pre-cooling the tissue prior to energy emission). In some embodiments, gel pad 728 is disposable and is replaced between treatment sessions and/or in between patients.

Additionally or alternately, applicator 700 is inserted into a thin balloon, which can be replaced between treatment sessions and/or in between patients.

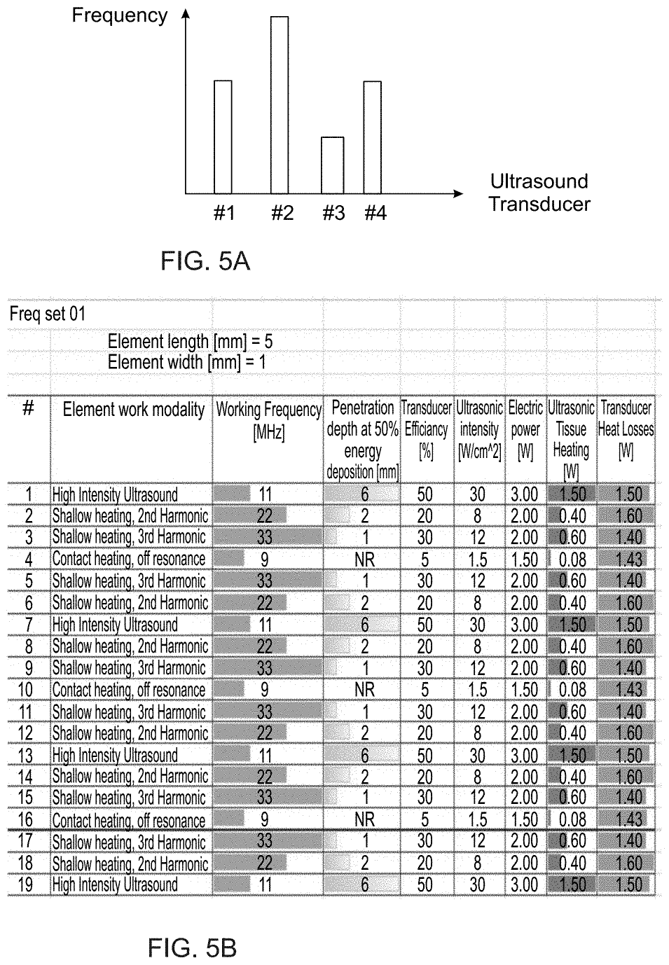

FIGS. 5A-B are exemplary graphs of activation of an array of ultrasound transducers, according to some embodiments.

In some embodiments, for example as schematically shown in FIG. 5A, various ultrasound transducers of an array are activated using different energy parameters sets (e.g. activated at different frequencies and/or at different intensities and/or at different durations and/or at different powers). In the schematic graph of FIG. 5A, 4 ultrasound transducers are activated at different frequencies. Optionally, activation is controlled to control heating of the tissue surface. Optionally, activation is controlled for reducing temperature differences between adjacent transducers.

Optionally, different transducers are activated with different parameter sets to produce a selected temperature distribution at deeper layers of the tissue.

FIG. 5B is a table of activation parameters of an array comprising a plurality of transducers, in this example including 19 transducers. In some embodiments, a higher or lower number of transducers may be used, for example between 1-50 transducers.

The exemplary parameters shown herein may be applied for treating skin tissue (e.g. for a skin tightening treatment).

In the described example, dimensions of a PZT element of each transducer included a rectangular emitting surface having a surface area of 5 mm\hat{ }2, for example having a length of 5 mm and a width of 1 mm. It is noted that PZT elements of other shapes and/or dimensions may be used.

In some embodiments, use of thin transducers (e.g. transducers having a width of less than 4 mm, less than 2 mm, less than 1 mm) provides for arranging a plurality of transducers adjacent each other to form an array.

Optionally, two or more transducers of the array are activated simultaneously to emit unfocused ultrasound for targeting a plurality of spaced apart tissue regions.

A potential advantage of using thin transducers that emit unfocused ultrasound may include the ability to treat a plurality of tissue regions using an array of transducers that is small enough to be mounted on a head of a hand held applicator. This may provide an advantage over, for example, focused ultrasound, in which a single large transducer may be needed for focusing the energy towards a single focal point.

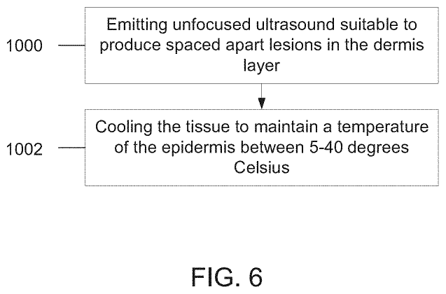

FIG. 6 is a flowchart of a method for aesthetic ultrasound skin treatment, according to some embodiments.

In some embodiments, ultrasound energy is emitted to produce spaced apart lesions of thermal damage, for example in the dermis layer of the skin (1000). In some embodiments, the energy is unfocused.

In some embodiments, the applied energy raises a temperature of defined volumes of dermis tissue, for example to a temperature between 60-70 degrees Celsius. Optionally, the thermally damaged volumes are configured a distance below the uppermost skin layer, the epidermis, for example a distance of at least 1 mm, at least 1.5 mm, at least 2 mm, at least 3 mm, at least 5 mm or intermediate, longer or shorter distances.

In some embodiments, cooling is applied to maintain a temperature of the epidermis between 5-40 degrees Celsius, such as 5-10 degrees, 10-20 degrees, 7-15 degrees, 20-30 degrees, or intermediate, higher or lower ranges. Optionally, the applicator's cooling module is set to a temperature of between −5 to -20, effective to reach the 5-40 degrees range on the tissue surface. In some embodiments, cooling of the epidermis to a temperature below 1 degree Celsius is avoided, for example to prevent a situation in which the skin adheres to the applicator.

In some embodiments, cooling is applied prior to energy emission.

Additionally or alternatively, cooling is applied in between periods of energy emission. Additionally or alternatively, cooling is applied during energy emission.

Optionally, the transducer's surface is continuously cooled. In some embodiments, cooling is applied in response to a temperature indication, for example if a temperature indicated by one or temperature sensors contacting the tissue is higher than a threshold, for example higher than 20 degrees, higher than 30 degrees, higher than 40 degrees or intermediate, higher or lower thresholds, stronger cooling is applied. Optionally, activation of the TEC element is controlled in accordance with the temperature indication.

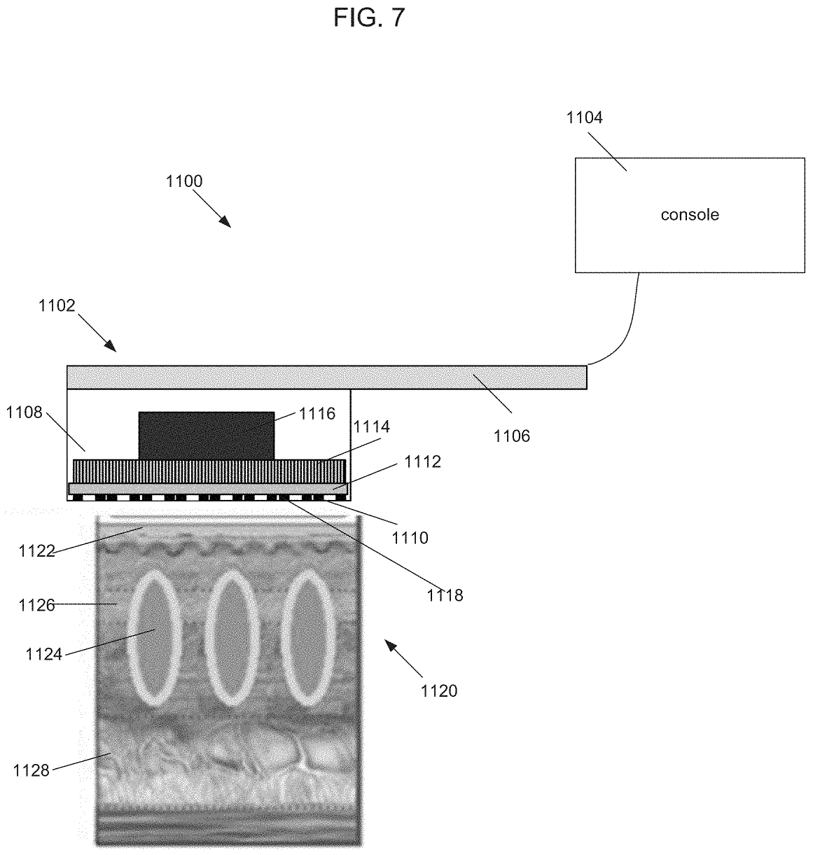

FIG. 7 is a schematic diagram of a system for ultrasound skin treatment, according to some embodiments.

In some embodiments, system 1100 comprises a hand unit 1102 operably coupled to a console 1104. In some embodiments, hand unit 1102 is coupled to the console by a wired connection. Additionally or alternatively, hand unit 1102 is coupled to the console by a wireless connection.

In some embodiments, hand unit 1102 comprises a handle 1106 to which an ultrasound applicator 1108 is attached. In some embodiments, applicator 1108 comprises one more energy emitting elements, such as transducers 1110. In some embodiments, applicator 1108 comprises a cooling module, for example comprising a TEC element 1112; a heat sink 1114; and optionally a fan 1116. In some embodiments, one or more temperature sensors 1118 are incorporated in the applicator, for example positioned in proximity and/or on transducers 1110.

In some embodiments, applicator 1108 is positioned on the treated skin 1120.

Optionally, the applicator is positioned directly, for example such that the energy emitting surfaces of the transducers 1110 contact the skin directly.

Alternatively, gel is applied the skin. In some embodiments, a gel blister is coupled to applicator 1108. Optionally, the gel blister is configured for slow release of the gel to apply it to the skin, for example during treatment.

Alternatively, applicator 1108 is inserted into a thin balloon which in turn contacts the skin.

In some embodiments, during operation, handle 1106 is moved across a surface of epidermis 1122, for example by a physician. In some embodiments, the movement pattern is selected in accordance with the intended lesion pattern in the tissue. In some embodiments, movement is performed in a direction substantially perpendicular to the long axes of the transducers. Alternatively, movement is performed in a direction substantially parallel to the long axes of the transducers.

Alternatively, movement is performed in a direction substantially at an angle to the long axes of the transducers.

In some embodiments, a shape and/or size of the transducer's emitting surface and/or a manner in which the transducer is moved across the tissue surface are selected to produce a certain lesion pattern, for example movement of a rectangular transducer across the tissue, along the long axis of the transducer may produce continuous, spaced apart lines of strong thermal damage inside the tissue. Alternatively, movement of the rectangular transducer along its short axis may produce continuous, close lines of relatively weak thermal damage inside the tissue.

In some embodiments, a squared, circular, or semi-circular transducer surface having a maximal width of, for example, 2 mm, is moved intermittently across the tissue surface (e.g. with 3-10 second intervals between emissions) to generate spaced apart points of thermal damage with undamaged tissue between them, in a fractional manner.

In some embodiments, moving the handle while setting a predetermined delay between excitation pulses of the plurality of ultrasound elements of the applicator provides for steering the emitted beam through a range of angles, to produce a desired thermal effect in the tissue.

In some embodiments, applicator 1108 is held against the tissue and energy is emitted for a time period of between 1-30 seconds, such as 3 seconds, 5 seconds, 9 seconds, 10 seconds, 20 seconds or intermediate, longer or shorter time periods before moving the applicator again to another location. Optionally, the emission duration and/or other energy parameters are selected in accordance with the tissue type and/or condition to be treated. For example, for treating wrinkles in the forehead each energy emission period may range between 8-10 seconds. When treating sagging skin in the neck area, the energy emission period may be longer, for example between 10-20 seconds. Optionally, the energy frequency is modified, for example a lower frequency is selected.

In some embodiments, energy is applied intermittently, for example with time intervals between 5-30 seconds between emission periods. Optionally, energy is applied in a duty cycle of between 1-50%. Alternatively, energy is applied in a continuous mode.

In some embodiments, one or more lesions 1124 are created in the tissue, for example in the reticular dermis 1126. Optionally, multiple lesions are created simultaneously (e.g. by using an array of transducers).

In some embodiments, a cross section profile of lesion 1124 comprises an elongated, substantially elliptical profile. In some embodiments, a volume of lesion 1124 is between 1 mm³ to 3 mm³, between 0.3 mm³ to 2 mm³, between 1 mm³ to 7 mm³ or intermediate, larger or smaller volume.

In some embodiments, lesions 1124 are spaced apart from each other, for example a distance 1128 of 1 mm, 2 mm, 4 mm, 6 mm, 8 mm or intermediate, longer or shorter distances. In some embodiments, damaged tissue within the lesion comprises denatured collagen and/or cells that underwent necrosis and/or coagulated blood. In some embodiments, the damage induces an inflammatory wound-healing response of the tissue.

In some embodiments, tissue between the lesions remains substantially undamaged. A potential advantage of healthy tissue between the lesions of thermal damage may include stimulating growth of tissue, such as collagen and/or elastin fibers, which in turn may lead to remodeling of the tissue, lifting and/or tightening the skin. In some cases, a visible effect on the skin may observed after 1 months, after 3 months, after 6 months, after 9 months or intermediate, longer or shorter time periods.

In some embodiments, epidermis 1122 remains substantially undamaged.

Alternatively, in some embodiments, minor thermal damage is caused to the epidermis. Optionally, the damage is higher towards the bottom of the epidermis, closer to the dermis, and lower towards the uppermost external surface of the dermis.

In some embodiments, tissue layer 1128 comprising fat and/or connective tissue define as a natural barrier of the thermal damage. In some embodiments, due to higher energy attenuation in the dermis as compared to energy attenuation in fat tissue, the unfocused ultrasound heats the dermis to a substantially higher temperature than the temperature in the fat tissue. Optionally, in such a setup, subcutaneous fat of the hypodermis defines a lower limit to the spatial spread of the thermal damage. A potential advantage of using unfocused ultrasound may include that the dermis is targeted regardless of anatomical variations, such as variations in a depth of the dermis and/or presence of wrinkles. This reduced sensitivity to anatomical variations may provide an advantage over, for example, focused ultrasound, in which a fixed focal point has to be predetermined and the energy may reach undesired tissue locations if the anatomy of the tissue is slightly different than the one taken into consideration.

In some embodiments, a contact between the applicator and the tissue surface (e.g. the epidermis) is assessed. In some embodiments, one or more of the following may be used for indicating contact with the tissue (e.g. whether contact has been established and/or whether the applicator is positioned in sufficient proximity to the tissue):

A. measuring a change in impedance of the one or more transducers (e.g. prior to contact and following contact with the tissue)

B. measuring electric power consumption of the one or more transducers, before, during and/or after excitation.

C. measuring a change in ringing attenuation of the one or more transducers following excitation

D. measuring a change in a cooling curve of the one or more transducers following excitation

E. measuring bio-impedance of the tissue, for example via two transducers

F. measuring changes in a cooling curve of one or more temperature sensors before and/or during excitation

G. measuring changes in acoustic signals received by one or more transducers

H. measuring a change in amplifier gain

I. measuring a change in the capacitance of the one or more transducers

J. measuring capacitance differences between upper electrodes of different transducers, such as adjacent transducers.

In some embodiments, overheating of the transducer is reduced or prevented.

Optionally, a temperature of the emitting surface of the transducer is maintained below 20 degrees Celsius, 25 degrees Celsius, 15 degrees Celsius or intermediate, higher or lower temperatures.

In some embodiments, overheating of the tissue surface is reduced or prevented. Optionally, a temperature of the tissue surface is maintained below, for example, 40 degrees, 38 degrees, 41 degrees. In some embodiments, one or more of the following may be used for assessing a temperature of the transducer and/or the tissue:

A. measuring a temperature of the ultrasound emission element (e.g. PZT), for example before, during and/or following energy emission, for example using one or more temperature sensors.

B. measuring a capacitance of the ultrasound emission element as an indicator of temperature.

C. measuring a temperature of a coating disposed on the one or more transducers, for example using one or more temperature sensors positioned adjacent the coating and/or via thermo-resistors incorporated in circuitry embedded in the coating, in accordance with some embodiments.

D. measuring bio-impedance of the tissue and/or changes thereof.

E. measuring impulse response damping changes and assessing a temperature of the transducer based on a correlation between damping changes and temperature.

F. measuring impedance of the transducer.

Optionally, temperature sensitive materials are incorporated in the transducer, and a change in their properties affects the transducer's impedance. For example, viscosity of glue coupling a PZT element to the base changes in response to a change in temperature of the PZT element.

In some embodiments, one or more of the following may be used for reducing pain before and/or during and/or following treatment:

A. Exciting one or more transducers at a low frequency, for example a frequency between 50-400 KHz, such as 90KHz 100 KHz, 200 KHz, 300 KHZ.

Optionally, the intensity is selected to be between 0.05 W/cm\hat{ }2 to 1 W/cm\hat{ }2.

In some embodiments, the low frequency is obtained by activating two or more adjacent transducers at close but not similar frequencies, to produce an acoustic beat.

Additionally or alternatively, the transducer is activated at a bending mode frequency.

B. Exciting one or more transducers at their bending mode resonance frequencies. Optionally, extensive cooling is applied simultaneously.

C. Exciting two or more adjacent transducers at slightly different frequencies that are close enough to each other to generate an acoustic beat, for example using frequencies in the range of 50-200 KHz, according to some embodiments.

D. Prior to treating, activating one or more transducers at an intensity that is higher than the intensity required for treatment, for a short period of time, to numb nerves at the targeted area. Optionally, partial blocking of pain is achieved by producing a low level of thermal damage in the tissue, as nerves are more sensitive to the high temperatures as compared to non-nervous tissue. In an example, energy is applied over 0.01-1 second at an intensity that is between 20-200% higher than the treatment intensity, to cause numbing of nerves. Optionally, the energy is applied as a pulse train.

### Exemplary System

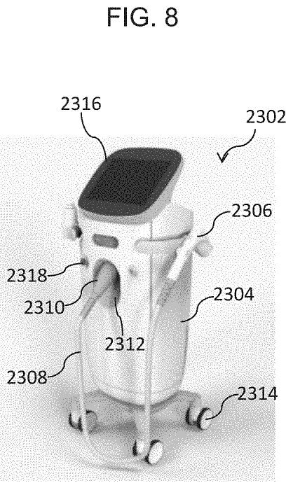

According to some exemplary embodiments, a system for delivery of ultrasonic waves, for example non-focused ultrasonic waves for skin treatments comprises an ultrasound applicator and a control console. In some embodiments, the system is configured to deliver ultrasonic waves to selected facial and/or neck regions, for example submental regions of the neck. Reference is now made to FIG. 8 depicting a system for delivery of ultrasonic waves for skin treatments, according to some exemplary embodiments of the invention.

According to some exemplary embodiments, a system for delivery of ultrasonic waves, for example system 2302 comprises an ultrasound applicator 2306, for example a handheld applicator, connected by an elongated cable 2308, for example an elongated flexible cable, to a control console 2304. In some embodiments, the applicator 2306 comprises two or more ultrasound transducers configured to be in contact or near an external surface of a skin. Optionally, the two or more ultrasound transducers are arranged in an array of ultrasound transducers. In some embodiments, the applicator 2306 is shaped and sized to be placed in contact with facial and/or neck regions, for example submental neck regions. In some embodiments, each transducer comprises an active surface for generating and delivering of ultrasonic energy. In some embodiments, a surface area of said active surface is in a range of 3 mm² -9 mm², for example 3 mm² -5 mm², 4 mm² -8 mm², 6 mm² -9 mm² or any intermediate, smaller or larger range of values.

According to some exemplary embodiments, the ultrasound applicator is shaped and sized to deliver ultrasonic energy to one or more skin regions having a surface area size in a range of 5 cm²-100 cm², for example facial and/or neck skin regions.

According to some exemplary embodiments, the applicator 2306 comprises a temperature control unit configured to cool a surface of the transducers placed in contact with the skin surface and/or regions between the transducers that contact the skin surface. In some embodiments, the temperature control unit is configured to cool the transducers array. In some embodiments, the temperature control unit, comprises one or more thermoelectric coolers (TECs). In some embodiments, each TEC comprises a cold surface and a hot surface.

According to some exemplary embodiments, the temperature control unit comprises at least one heat conductor, shaped and sized to contact the transducers array and a cold surface of the one or more TECs. In some embodiments, the at least one heat conductor is made from Aluminum or from any other heat conducting material. In some embodiments, the at least one heat conductor is configured to conduct heat from the transducers array, for example from the transducers and/or from regions between the transducers to the cold surface of the one or more TECs. Alternatively or additionally, the at least one heat conductor is configured to conduct cold from the cold surface of the one or more TECs to the transducers array, for example to the transducers and/or to regions between the transducers.

According to some exemplary embodiments, the temperature control unit comprises a cooling fluid chamber located within the applicator and contacting a hot surface of the one or more TECs. In some embodiments, a wall of the cooling fluid chamber which is placed in contact with the hot surface is configured to conduct heat from the hot surface of the one or more TECs to a cooling fluid, for example water, within the cooling fluid chamber. In some embodiments, the cooling fluid circulates between the applicator and a cooling module of the console 2304. In some embodiments, the cooling fluid flows within a flow path, for example one or more cooling fluid tubes or channels within the elongated cable 2308 into the cooling module, and from the cooling module to the cooling fluid chamber of the applicator 2306.

According to some exemplary embodiments, the temperature control unit comprises one or more temperature sensors, for example thermistors, are located near and/or placed in contact with the transducers and/or with regions between the transducers, for example to sense the temperature of the transducers and/or near the transducers. Alternatively or additionally, the one or more temperature sensors are placed in contact with the skin, for example to sense the temperature of the skin before, during and/or after delivery of ultrasonic waves to the tissue.

According to some exemplary embodiments, the applicator 2306 comprises an applicator user interface, configured to deliver one or more indications, for example human detectable indications to a user of the system. In some embodiments, the indications comprise visual indications visible to a user holding the applicator during a treatment. In some embodiments, the applicator user interface is configured to receive input from a user of the system 2302 and/or from a user holding the applicator 2306, for example during a treatment. In some embodiments, the applicator user interface comprises one or more buttons and/or switches configured to receive the input from the user.

According to some exemplary embodiments, the applicator 2306 is a replaceable applicator that can be replaced according to a selected treatment and/or according to a selected treatment region. In some embodiments, the applicator 2306 is connected to the elongated cable 2308 by a connector configured to allow easy detachment of the applicator from the elongated cable 2308. In some embodiments, different applicators vary in one or more of size and/or shape of the applicator, number of transducers, transducers arrangement in an array, type of transducers, applicator surface area size and/or shape configured to be placed in contact with the skin and/or cooling capacity of the applicator.

According to some exemplary embodiments, the elongated cable 2308 comprises one or more flow paths, for example one or more tubes or channels, between the control console and the applicator. In some embodiments, as described previously, the one or more flow paths are shaped and sized to allow circulation of cooling fluid between the control console 2304 and the applicator 2306. Additionally, the elongated cable 2308 comprises electrical wiring for conducting electricity between the control console 2304 and the applicator 2306.

According to some exemplary embodiments, the console 2304 comprises a holder configured to hold the applicator 2306, for example when the applicator 2306 is not in use. In some embodiments, the console 2304 and the system 2302 are mobile. In some embodiments, the console 2304 comprises one or more wheels 2314 configured to allow easy movement of the console 2304 on a surface. In some embodiments, the console 2304 is shaped as a tower. In some embodiments, a maximal height of the console from a surface is in a range of 40-160 cm, for example 40-100 cm, 70-130 cm, 90-160 cm or any intermediate, smaller or larger range of values.

According to some exemplary embodiments, the console 2304 comprises a control circuitry and memory. In some embodiments, the memory stores one or more treatment protocols or values of parameters thereof, for example ultrasonic waves frequency values, ultrasonic waves intensity values, total ultrasonic energy values, cooling-related parameters values, treatment session duration, treatment duration per selected treated region, cooling duration during and/or after ultrasonic energy application. In some embodiments, the memory stores treatment parameter values per a treatment session, for example a treatment session of one or more facial and/or neck regions. Alternatively or additionally, the memory stores treatment parameter values per a treated region, for example per facial and/or neck treated regions. In some embodiments, the memory stores treatment parameter values per skin type and/or skin color. In some embodiments, the control circuitry activates the two or more transducers of the applicator according to the at least one treatment protocol and/or parameters thereof stored in the memory.

According to some exemplary embodiments, the console 2304 comprises a user interface configured to deliver one or more indications to a user of the system 2302. Additionally, the user interface is configured to receive input from a user of the system 2302. In some embodiments, the user interface comprises a display, for example display 2316. In some embodiments, the display 2316 is configured to present one or more visual indications and/or alerts to a user, for example indications related to the system function, treatment, treatment parameter values. Alternatively or additionally, the display 2316 is configured to deliver one or more visual indications and/or alerts related to temperature levels of the applicator, transducers in the applicators and/or temperature levels of the treated body regions.

According to some exemplary embodiments, the display 2316 displays information regarding at least one of type of ultrasound applicator currently connected to the console or type of ultrasound applicator used in a previous treatment session, number of ultrasound transducers, intensity of ultrasound energy in W/cm² per one or more ultrasound transducers, amount of ultrasound energy in Joules. In some embodiments, the display 2316 displays the ultrasound energy derived to the tissue in at least one of a pulse, a series of pulses, a treatment session, in all or some previous treatment sessions, and/or for a planned treatment.

According to some exemplary embodiments, the display 2316, for example a touch sensitive display, is configured to receive input from a user, for example selection of treatment protocols, selection of treatment parameter values, and/or selection of system operation parameter values. In some embodiments, the input received by the user interface comprises patient-related input, for example patient details, patient profile, personalized treatment protocols and/or parameters thereof. In some embodiments, the display 2316 comprises a movable display configured to move in order to adjust an angle of the display to a point of view of a user of the system, for example a sitting or a standing user.

According to some exemplary embodiments, the user interface comprises one or more buttons and/or switches configured to receive input from a user of the system, for example during treatment. In some embodiments, the one or more buttons comprises an emergency button, for example emergency button 2318, configured to stop the delivery of ultrasonic energy or the activation of the system, for example if the temperature of the transducers and/or the treated regions is higher than a predetermined value. In some embodiments, the user interface comprises a foot switch or is connected to an external foot switch, configured to activate and/or deactivate the ultrasonic transducers or any other component of the system 2302 by a foot of a user.

According to some exemplary embodiments, the console 2304 comprises a port, for example port 2312, configured to allow connection between a connector 2310 of the elongated cable 2308 and the console 2304. In some embodiments, the port 2312 comprises one or more electrical and/or cooling fluid flow path connectors configured to allow connection of the electrical wiring and/or cooling fluid flow path in the elongated cable 2308 to the console 2304.

According to some exemplary embodiments, the control circuitry of the console 2304 receives signals from one or more sensors and/or components in the applicator regarding skin temperature, ultrasound coupling efficiency and/or energy deposition into the skin. In some embodiments, the energy deposition into the skin is measured based on the coupling of the ultrasound applicator, for example at least some or all of the ultrasound transducers, to the skin.

### Exemplary Treatment Procedure

According to some exemplary embodiments, ultrasonic energy is delivered to skin tissue, for example to one or more of facial tissue, neck tissue and/or submental tissue. In some embodiments, the ultrasonic energy is delivered to one or more regions of the tissue, for example in a single treatment session. In some embodiments, the ultrasonic energy is delivered to the tissue, for example to allow reshaping of the tissue. In some embodiments, tissue reshaping comprises improving the appearance of wrinkles, for example wrinkles in the facial, neck and/or submental regions. In some embodiments, a maximal duration of a treatment session for treating facial, neck and/or submental skin is up to 60 minutes, for example up to 50 minutes, up to 40 minutes, up to 35 minutes, up to 30 minutes, up to 20 minutes or any intermediate, shorter or longer time duration.

According to some exemplary embodiments, the ultrasonic energy is delivered to the skin in pulses of at least 3 Joules, for example at least 3 Joules, at least 4 Joules, at least 5 Joules, at least 6 Joules, at least 7 Joules, at least 8 Joules or any intermediate, smaller or larger value. In some embodiments, the delivered ultrasonic energy heats tissue layers in a depth of at least 1 mm from the external surface of the skin, for example at least 1.5 mm, at least 2 mm or any intermediate, smaller or larger depth, to a temperature of at least 45° C., for example at least 50° C., at least 55° C., or any intermediate, smaller or larger temperature level.

According to some exemplary embodiments, the ultrasonic energy is delivered to the skin tissue of a single target region having a surface area size which is equal to a footprint of the transducers array of the applicator, for example to surface size of at least 20 mm×5 mm, in one or more pulses, or in at least 10 pulses, for example at least 20 pulses, at least 30 pulses, at least 50 pulses, at least 70 pulses, at least 100 pulses or any intermediate, smaller or larger number of pulses. In some embodiments, a duration of each pulse of ultrasonic energy is at least 0.5 seconds, for example 1 second, 2 seconds, 4 seconds, 5 seconds, 7 seconds, 10 seconds or any intermediate, smaller or larger time duration.

According to some exemplary embodiments, parameter values of the delivered ultrasonic energy are selected to get a reduction of at least 10% in a depth of wrinkles, for example at least 15%, at least 20%, at least 50%, at least 60%, at least 70% reduction or any intermediate, smaller or larger percentage of wrinkles depth reduction, after a time period of at least 1 week, for example at least 2 weeks, at least 3 weeks or any intermediate, shorter or longer time duration.

According to some exemplary embodiments, parameter values of the delivered ultrasonic energy are selected to achieve a reduction of at least 1 point, at least 2 points, at least 3 points or any intermediate, smaller or larger number of points in wrinkle severity scales comprising the Wrinkle Severity Rating Scale (WSRS), the Glogau scale, the Fitzpatrick wrinkle scale, the Fitzpatrick wrinkle score, and/or the Fitzpatrick Wrinkle and Elastosis Scale (FWES), after a period of at least 1 week from the treatment, for example compared to a baseline score. In some embodiments, the parameter values are selected by reading one or more parameter values from a table, for example a lookup table, stored in a memory.

According to some exemplary embodiments, the ultrasonic energy is delivered to heat one or more tissue layers located at a depth of 1 mm-2.5 mm from an external surface of the skin, for example tissue layers at a depth of 1 mm-2 mm, 1.5 mm-2.5 mm or any intermediate, smaller or larger range of depths from the external surface of the skin. In some embodiments, the delivered ultrasonic energy heats the tissue layers at a depth 1 mm-2.5 mm from an external surface of the skin to a temperature of at least 45 degrees Celsius, for example at least 50 degrees Celsius, at least 55 degrees Celsius or any intermediate, smaller or larger temperature value.

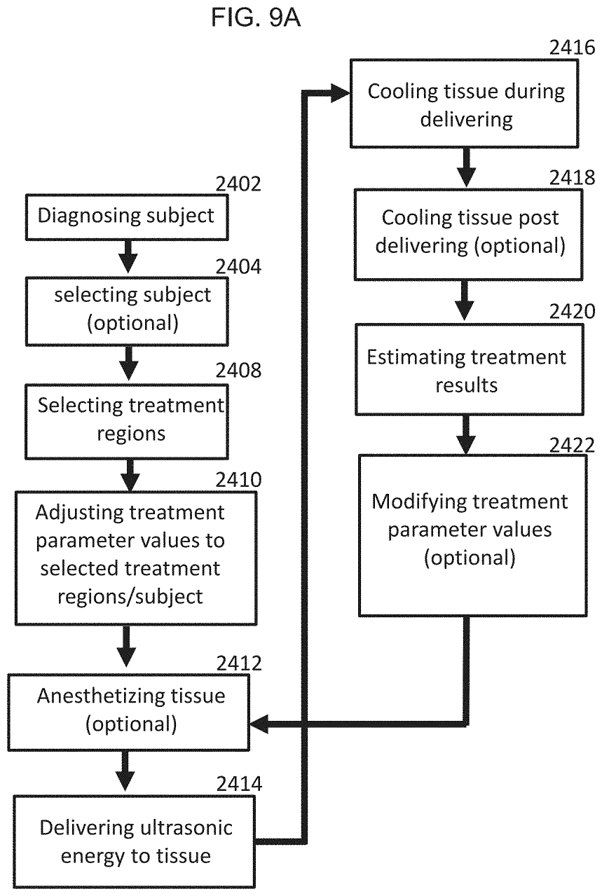

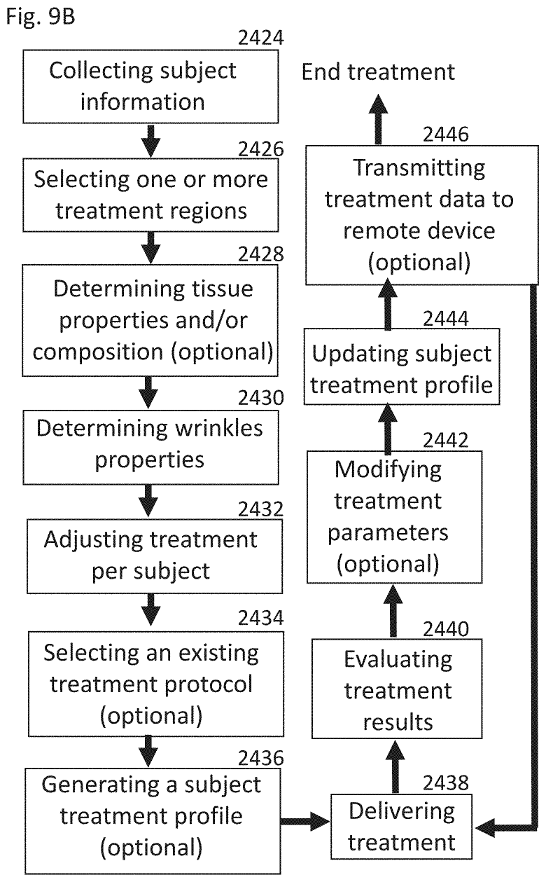

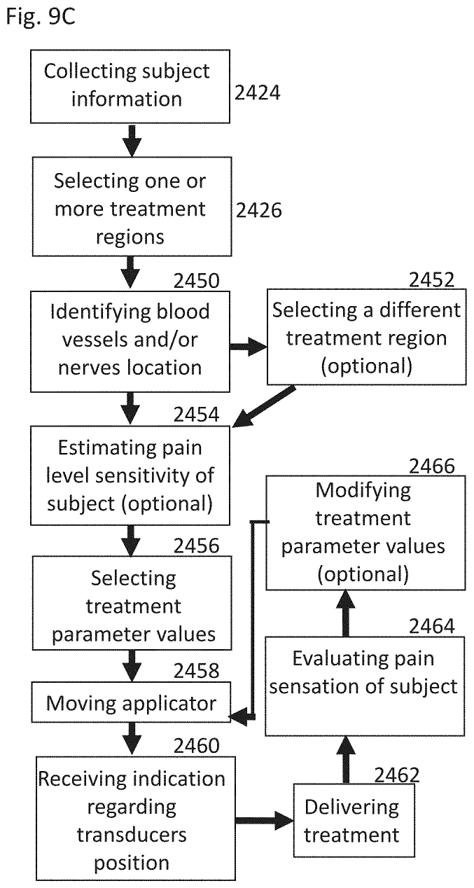

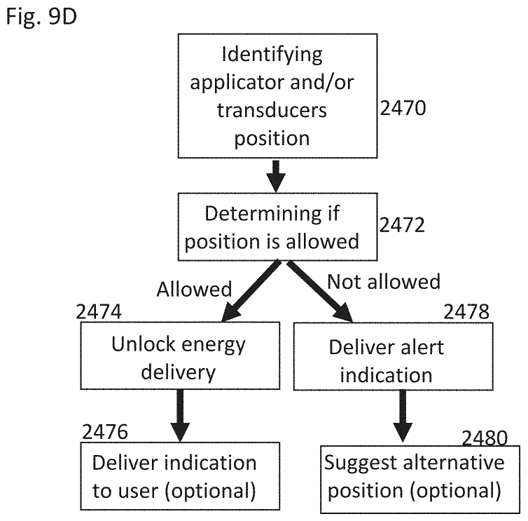

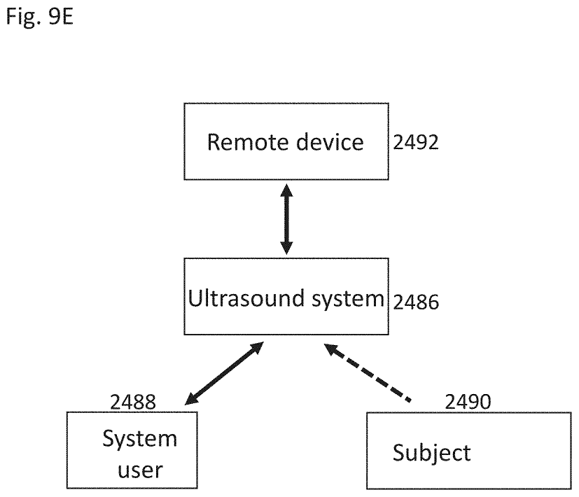

According to some exemplary embodiments, the ultrasonic energy is delivered to facial and/or neck skin tissue regions, for example to promote one or more of brow elevation, increase upper lid show, decrease in submental fullness, decrease in neck wrinkles, and/or decrease in facial wrinkles. Reference is now made to FIG. 9A, depicting a process for delivering of ultrasonic waves to skin tissue, for example to skin tissue in facial and/or neck regions, according to some exemplary embodiments of the invention.

According to some exemplary embodiments, a subject is diagnosed at block 2402. In some embodiments, an expert, for example a physician or a cosmetician diagnoses a condition of the subject's skin. In some embodiments, the expert diagnoses the condition of the skin in facial, neck and/or submental regions. In some embodiments, diagnosing a skin condition comprises determining wrinkle severity, for example using wrinkle severity scales. In some embodiments, the wrinkle severity scales comprise the Wrinkle Severity Rating Scale (WSRS), the Glogau scale, the Fitzpatrick wrinkle scale, the Fitzpatrick wrinkle score, and/or the Fitzpatrick Wrinkle and Elastosis Scale (FWES). In some embodiments, a baseline a baseline score of one or more of the wrinkle severity scales is determined.

According to some exemplary embodiments, during subject diagnosis at block 2402, the subject skin type, for example skin phototype is classified. In some embodiments, the skin phototype of a subject is classified, for example according to the Fitzpatrick skin type scale.

According to some exemplary embodiments, during subject diagnosis at block 2402, tissue composition, for example the percentage and/or existence of fat tissue, connective tissue, scar tissue or any other tissue type that may affect the delivery of ultrasonic energy, is determined. In some embodiments, the tissue composition at selected one or more regions is determined.

According to some exemplary embodiments, a subject is selected to undergo the treatment at block 2404. In some embodiments, the subject is selected based on the results of the diagnosis performed at block 2402.

According to some exemplary embodiments, one or more treatment regions are selected at block 2408. In some embodiments, the one or more selected treatment regions comprise facial regions and/or neck regions, for example submental regions. In some embodiments, the one or more treatment regions are selected based on the results of the diagnosis performed at block 2402.

According to some exemplary embodiments, values of at least one treatment parameter are adjusted to a selected treatment region and/or per a specific selected subject at block 2410. In some embodiments, the at least one parameter comprise one or more of ultrasonic waves frequency, ultrasonic waves intensity, amount of ultrasonic energy, total number of pulses of ultrasonic waves, number of ultrasonic waves pulses per a train of pulses, number of trains, overall duration of a treatment session, duration of a treatment per tissue region, cooling intensity during and/or following treatment, and/or cooling duration following treatment.

According to some exemplary embodiments, the treatment parameters comprise one or more of the number of pulses of energy applied on the tissue, the acoustic intensity in W/cm-2 per pulse, the number of transducers used for generating the energy pulse, the duration of each pulse, the total area of the transducers used for generating the energy pulse, the length of each transducer, the width of each transducer and/or the footprint area of the applicator.

According to some exemplary embodiments, the treatment parameter values are adjusted according to one or more of the wrinkles classification, wrinkles scale score, skin phototype and/or tissue composition at the selected treatment regions, In some embodiments, the treatment parameter values are adjusted according to one or more subject-related parameters, for example clinical condition of the subject, medical history of the subject, current drug regime of the subject, subject age, and/or subject gender.

According to some exemplary embodiments, values of at least one treatment parameter are adjusted according to a sensitivity of the subject to pain. Additionally or alternatively, values of at least one treatment parameter are adjusted according to the likelihood of the subject to develop erythema and/or edema during and/or following the treatment.

According to some exemplary embodiments, the tissue is optionally anesthetized, for example locally anesthetized, at block 2412. In some embodiments, tissue at the selected treatment regions is locally anesthetized. In some embodiments, the tissue is locally anesthetized to induce local analgesia during and/or following the treatment. In some embodiments, the tissue is topically anesthetized, for example by topically applying an anesthetizing gel. Alternatively or additionally, the tissue is anesthetized by local injections of an anesthetizing drug. In some embodiments, the anesthetizing compound comprises one or more of lidocaine, tetracaine, prilocaine, cream or gel, for example EMLA cream.

According to some exemplary embodiments, ultrasonic energy is delivered to tissue, for example skin tissue at block 2414. In some embodiments, the ultrasonic energy is delivered by ultrasonic waves generated by an array of transducers placed in contact with the external surface of the skin tissue. In some embodiments, the external surface of the transducers array is placed in direct contact with the external surface of skin at the selected treatment regions. Alternatively, the external surface of the transducers or the transducers array is placed in indirect contact with the external surface of the skin, for example via a cover placed between the transducers array and the skin.

According to some exemplary embodiments, the ultrasonic energy is delivered to the skin tissue, for example to the skin at the selected treatment regions, up to a total energy level of 1-6 Joules per pulse of ultrasonic energy, for example 1-3 Joules, 2-5 Joules, 4-6 Joules or any intermediate, smaller or larger range of values. In some embodiments, the ultrasonic energy is delivered during a maximal time period of up to 60 minutes, for example up to 50 minutes, up to 40 minutes, up to 30 minutes, up to 20 minutes or any intermediate, shorter or longer time period for a single treatment session.

According to some exemplary embodiments, each of the selected treatment region receives pulses of ultrasonic energy generated by a plurality of transducers. In some embodiments, the pulses are delivered to the tissue in one or more repetitions In some embodiments, the number of pulses per treatment region is in a range of about 5-100 pulses, for example about 5-30 pulses, about 20-50 pulses, about 40-100 pulses or any intermediate, smaller or larger number of pulses.

According to some exemplary embodiments, the number of pulses varies according to the treatment region. In some embodiments, for example when treating regions located at the side of the face, the number of pulses per a single repetition is in a range of about 10-60, for example about 10-40 pulses, about 20-50 pulses, about 35-60 pulses or any intermediate, smaller or larger number of pulses. In some embodiments, for example when treating regions above and/or near the brows, the number of pulses per a single repetition is in a range of about 8-70, for example about 8-30 pulses, about 20-60 pulses, about 50-70 pulses or any intermediate, smaller or larger number of pulses. In some embodiments, for example when treating regions of the upper neck and/or submental regions, the number of pulses per a single repetition is in a range of about 7-70, for example about 7-40 pulses, about 20-60 pulses, about 40-70 pulses or any intermediate, smaller or larger number of pulses.

According to some exemplary embodiments, for example when treating the right side of the upper neck, the number of pulses per a single repetition is in a range of about 10-70 pulses, for example about 10-40 pulses, about 30-60 pulses, about 35-70 pulses or any intermediate, smaller or larger number of pulses. In some embodiments, for example when treating the left side of the upper neck, the number of pulses per a single repetition is in a range of about 7-40 pulses, for example about 7-20 pulses, about 15-30 pulses, about 25-40 pulses or any intermediate, smaller or larger number of pulses.

According to some exemplary embodiments, the acoustic output intensity of each of the transducers is in a range of 5-40 W/cm\hat{ }2, for example 5-15 W/cm\hat{ }2, 10-25 W/cm\hat{ }2, 20-40 W/cm\hat{ }2 or any intermediate, smaller or larger range of values. In some embodiments, an exemplary calculation of the ultrasonic energy applied to the tissue by an ultrasound transducers array of 7 transducers operating at an acoustic output intensity of 25 W/cm\hat{ }2 for 4 seconds is: 25[W/cm\hat{ }2 (acoustic intensity)]*0.45[cm (active length)]*0.1[cm (active width)]*7[No. of transducers]*4[sec (pulse duration)]*(4.5*7 {total transducers area}/(25*8 {footprint area}))=˜5 Joules per a pulse duration of 4 seconds.

According to some exemplary embodiments, a link between the number of Joules per pulse, and the energy intensity in W/cm² depends on one or more of, number of transducers used and the time duration in which a specific energy intensity is emitted, for example pulse duration, pre-pulse cooling duration, post-pulse cooling duration, and ultrasonic frequency.

According to some exemplary embodiments, the ultrasonic energy is delivered in one or more pulses of unfocused ultrasonic energy. In some embodiments, the pulses are delivered through a surface area size in a range of 3 mm²-7 mm² or any intermediate, smaller or larger range of values. In some embodiments, each pulse has an intensity in a range of 5 W/cm²-60 W/cm², for example 10 W/cm²-30 W/cm² 10 W/cm²-17 W/cm², 15 W/cm²-25 W/cm², 20 W/cm²-30 W/cm², 25 W/cm²-50 W/cm², 40 W/cm²-60 W/cm² or any intermediate, smaller or larger range of values. In some embodiments, a time duration in which said ultrasonic energy is actively transmitted is in a range of 1-10 seconds per pulse, for example 1-5 seconds, 3-7 seconds, 6-10 seconds or any intermediate, smaller or larger range of values. In some embodiments, the unfocused ultrasonic energy is delivered to cover one or more skin regions having a surface area size in a range of 2 cm²-150 cm², for example 2 cm²-50 cm², 10 cm²-100 cm², 50 cm²-150 cm² or any intermediate, smaller or larger range of values, when optionally delivered from a fixed position. In some embodiments, the unfocused ultrasound energy is delivered by 1-20 transducers, for example a single transducers, 1-10 transducers, 5-15 transducers, 10-20 transducers or any intermediate, smaller or larger number of ultrasound transducers.

According to some exemplary embodiments, the ultrasonic energy is delivered with treatment parameter values adjusted to generate a high thermal effect of about 55-65° C. in tissue layers in a depth of about 0.5-6 mm, for example about 0.7-1.2 mm, about 0.9-1.5 mm, about 1-2 mm, about 2-4 mm, about 3-6 mm, about 2-6 mm or any intermediate, smaller or larger range of values, from the external surface of the skin. In some embodiments, the ultrasonic energy is delivered with treatment parameter values adjusted to generate a moderate thermal effect of about 47-55° C. in tissue layers in a depth of about 0.4-0.7 mm and about 2-2.5 mm from the external surface of the skin.

According to some exemplary embodiments, when heating a tissue layer located at a depth of at least 1.5 mm from the external surface of the skin with ultrasonic energy of 15 W/cm² the tissue layer is unaffected. In some embodiments, when gradually increasing the ultrasonic energy, for example to 20 W/cm², the tissue layer is affected and optionally a necrotic region is formed. In some embodiments, increasing the ultrasonic energy to levels higher than 25 W/cm², increases the volume and/or the size of the necrotic region. Optionally, the effect of energy levels higher than 15 W/cm² on the tissue, for example the increase in size and/or volume of the necrotic region is exponential.

According to some exemplary embodiments, a tissue contacting the transducers array is cooled during ultrasonic energy delivery at block 2416. In some embodiments, the tissue contacting the transducers, for example during the activation of the transducers, is cooled. Alternatively or additionally, the tissue contacting regions between adjacent transducers, for example during the activation of the transducers is cooled. In some embodiments, superficial tissue layers, for example tissue layers closer to the transducers array are cooled during the activation of the transducers. In some embodiments, superficial tissue layers comprise tissue layers up to a depth of 0.2 mm, for example up to a depth of 0.1 mm, up to a depth of 0.05 mm, or any intermediate, smaller or larger depth from the external surface of the skin.

According to some exemplary embodiments, the tissue contacting the transducers array is optionally cooled after the ultrasonic energy has been delivered at block 2418. In some embodiments, the tissue contacting the transducers array is cooled while the transducers generate ultrasonic waves and after they stop the ultrasonic waves generation. In some embodiments, the tissue contacting the transducers array is cooled for a period of about 1-10 seconds after stopping the activation of the ultrasound transducers, for example to reduce erythema and/or edema of the treated tissue.

According to some exemplary embodiments, the treatment results are estimated at block 2420. In some embodiments, the treatment results are estimated using one or more of the scales used to classify wrinkles, as described at block 2402. In some embodiments, the treatment results are estimated, for example as a change from a baseline value determined at block 2402. In some embodiments, the treatment results are estimated, for example whether one or more desired goals have been reached following the treatment.

According to some exemplary embodiments, one or more treatment parameter values are optionally modified at block 2422. In some embodiments, the one or more treatment parameter values are modified if the one or more desired goals have not been reached. In some embodiments, the treatment is repeated using the modified treatment parameter values.

### Exemplary Personalized Treatment

According to some exemplary embodiments, an ultrasound, for example a non-converging ultrasound skin treatment is personalized for a specific subject. In some embodiments, a personalized ultrasound skin treatment allows, for example, a more efficient treatment in optionally a shorter time period to reach a desired result relative to a non-personalized treatment. Reference is now made to FIG. 9B depicting a process for personalizing an ultrasound skin treatment, according to some exemplary embodiments of the invention.

According to some exemplary embodiments, information on a subject is collected at block 2424. In some embodiments, the collected information comprises personal information, for example age and/or gender. Additionally, or alternatively, the collected information comprises clinical information, for example at least one of medical history, family medical history, drug regime, and clinical data related to skin pathologies of the subject, and/or sensitivity to drugs.

According to some exemplary embodiments, one or more subjects are selected for the treatment so there is no therapeutic effect. In some embodiments, the treatment is a cosmetic treatment that cause no therapeutic effect.

According to some exemplary embodiments, at least one treatment region is selected at block 2426. In some embodiments, a treatment region is a region that has a surface size of up to 80 cm², for example up to 40 cm², up to 20 cm² or any intermediate smaller or larger surface area. In some embodiments, the at least one selected brain region comprises a facial or a neck region.

According to some exemplary embodiments, tissue properties and/or tissue composition at the selected treatment region are optionally determined at block 2428. In some embodiments, the tissue properties comprise mechanical properties of the tissue, the ability of the tissue to stretch, for example tissue elasticity, and/or comprises depth of a tissue volume to be heated by the ultrasonic energy from the skin surface. In some embodiments, the tissue composition comprises presence, thickness and/or size of tissue layers, for example fat tissue layers, located between the tissue surface and the tissue volume to be heated by the ultrasonic energy. Optionally, the tissue composition comprises presence of wound and/or scar tissue at the selected treatment region.

According to some exemplary embodiments, the ultrasound system, for example the ultrasound applicator comprises at least one pressure sensor and/or at least one sensor configured to measure the elasticity level of the skin surface upon application of pressure on the skin surface. In some embodiments, signals received from the elasticity sensor and/or from the pressure are used to determine the tissue properties at block 2428.

According to some exemplary embodiments, wrinkles properties are determined at block 2430. In some embodiments, properties of wrinkles at the selected treatment region are determined. In some embodiments, wrinkles properties comprise wrinkles length, wrinkles depth, for example average and maximal depth, wrinkles density, for example the number of wrinkles per surface area size.

According to some exemplary embodiments, the ultrasound treatment is adjusted according to the collected subject information, at block 2432. In some embodiments, at least one parameter of the treatment, for example values of the treatment parameter, is adjusted according to information collected at one or more blocks 2424, 2426, 2428 and 2430. In some embodiments, the at least one treatment parameter comprises energy intensity emitted from at least one ultrasound transducer, overall energy per treatment region in a selected time period, overall energy per a selected time period, duration of each energy pulse, overall energy per a treatment session and optionally additionally per treatment region.

According to some exemplary embodiments, an existing treatment protocol is selected at block 2434. In some embodiments, a plurality of treatment protocols are stored in a memory of the ultrasound system, or in a remote device. In some embodiments, at least some of the stored treatment protocols comprise different settings of treatment parameters. In some embodiments, at least one stored treatment protocol that matches the data collected from the subject is selected at block 2434.

According to some exemplary embodiments, a user profile is generated for a subject at block 2436. In some embodiments, the data collected from the subject, for example in at least one of the blocks 2424, 2426, 2428, and 2430 is stored in the user profile. Additionally or alternatively, the treatment parameter values selected for the skin treatment of the subject, and/or the selected treatment protocol are stored in the user profile.

According to some exemplary embodiments, an ultrasound skin treatment is delivered at block 2438. In some embodiments, the ultrasound skin treatment is delivered using the treatment parameter adjusted for the treated subject and/or according to the treatment protocol selected for the subject.

According to some exemplary embodiments, the treatment results are evaluated at block 2440. In some embodiments, the effect of the delivered treatment on the treatment target of the subject is evaluated at block 2440. In some embodiments, the evaluated treatment results comprise evaluated side effects, for example redness level of the skin, swallowing level of the skin, and/or pain sensation at the treated region of the skin. Additionally or alternatively, the evaluated treatment results comprise evaluation of skin tightening and/or evaluation of wrinkles at the treated region, for example depth, length and/or density of the wrinkles following treatment.

According to some exemplary embodiments, at least one treatment parameter is modified at block 2442. In some embodiments, the at least one treatment parameter is modified according to the treatment results evaluated at block 2440. In some embodiments, the at least one treatment parameter comprises number of treatment sessions and/or overall treatment duration needed to reach a desired treatment result.

According to some exemplary embodiments, a subject treatment profile is updated at block 2444. In some embodiments, the subject treatment profile is updated at block 2444 according to evaluated treatment results and/or modified treatment parameters.

According to some exemplary embodiments, treatment data is transmitted to a remote device, at block 2446. In some embodiments, the treatment data comprises at least one of data collected from the subject, at least one treatment parameter, evaluation of treatment results and/or at least some of the data stored in the subject treatment profile. In some embodiments, the remote device comprises a remote computer, a remote server, a remote cloud storage or any remote device that is configured to store data and/or to perform calculations on the stored data. In some embodiments, the transmitted data is used to generate a database, for example a big data database. In some embodiments, the transmitted data is transmitted without any personal identifying details of the subject, for example a country-issued ID number and/or a face picture of the subject.

### Exemplary Safety Considerations of Skin Treatment

According to some exemplary embodiments, the skin treatment is planned and/or is adjusted to be safe to the treated subject, for example not to cause nerve injury, blood vessels injury and/or to cause intolerable pain sensation. Reference is now made to FIG. 9C describing a process for modifying or selection of treatment parameters based on safety considerations.

According to some exemplary embodiments, subject information is collected at block 2424, and at least one treatment region is selected at block 2426, for example as described in FIG. 9B.

According to some exemplary embodiments, a location of undesired tissue, for example blood vessels and/or nerves is identified at block 2450. In some embodiments, the location of blood vessels and/or nerves at the selected treatment region is identified. In some embodiments, the location is identified using information collected by one or more imaging techniques, for example thermography, scanning ultrasound, computerized tomography (CT) and/or magnetic resonance imaging (MRI). Additionally or alternatively, the location of the blood vessels and/or nerves is identified using known anatomical information, and/or information collected from other subjects. Optionally, the location of blood vessels and/or nerves is identified using at least one transducer of the ultrasound transducers of the applicator.

According to some exemplary embodiments, if the select treatment target includes undesired tissue, for example blood vessels and/or nerves, at locations that may be affected by the treatment, then a different treatment region is selected at block 7452.

According to some exemplary embodiments, pain sensitivity of a subject is optionally estimated at block 2454. In some embodiments, sensitivity to pain caused by excess of heat or cold is estimated. In some embodiments, pain sensitivity is estimated using a test that generates heat, optionally known amount or degree of heat, followed by evaluation of the subject response. In some embodiments, sensitivity to pain is evaluated by delivery of a trial ultrasound treatment followed by evaluation of the subject response to the trial treatment. In some embodiments, in the trial treatment ultrasound energy is delivered with treatment parameters, for example energy intensity levels selected to generate a maximal tolerable pain sensation in a specific subject. Optionally, pain sensitivity is evaluated based on a subject report. In some embodiments, sensitivity to pain of a subject is quantitatively measured, for example, based on signals received from at least one electrode.

According to some exemplary embodiments, values of at least one treatment parameter are selected at block 2456. In some embodiments, the values are selected according to the identified location of blood vessels and/or nerves. Alternatively or additionally, the values are selected according to the estimated pain sensitivity level. In some embodiments, the at least one treatment parameter comprises energy intensity, duration of energy delivery, location of ultrasound transducers, cooling level of the transducers and/or skin surface contacting the transducers and/or angle of an emitting surface of the ultrasound transducers relative to the skin surface.

According to some exemplary embodiments, energy intensity is reduced in at least 5%, for example at least 10%, at least 20% or any intermediate, smaller or larger percentage value from the energy intensity level generated the maximal tolerable pain sensation, as identified at block 2456, for example using a trial ultrasound treatment.

According to some exemplary embodiments, the ultrasound applicator is moved at block 2458. In some embodiments, at least one ultrasound transducer is moved. In some embodiments, the ultrasound applicator is moved within the selected treatment region, for example along the skin surface.

According to some exemplary embodiments, an indication, for example an alert signal is received according to the detected position of the applicator and/or transducers, at block 2460. In some embodiments, the indication, for example an alert signal, is received based on a proximity of the applicator and/or transducers to the identified blood vessels and/or nerves.

According to some exemplary embodiments, ultrasound treatment is delivered at block 2462. In some embodiments, the ultrasound treatment is delivered when the applicator and/or transducers is at a location where treatment delivery will not affect identified blood vessels and/or nerves, or that the treatment will cause tolerable effect.

According to some exemplary embodiments, pain sensation of the subject is evaluated at block 2464. In some embodiments, the pain sensation is evaluated during and/or following the delivery of ultrasonic energy. In some embodiments, pain sensation is evaluated at least partly by receiving information on the temperature of the skin surface, for example skin surface contacting the transducers and/or transducers temperature, during and/or following energy delivery.

According to some exemplary embodiments, values of at least one treatment parameter are optionally modified at block 2466 according to the pain sensation evaluation. In some embodiments, the energy intensity is reduced in at least 5%, for example at least 10%, at least 20% or any intermediate, smaller or larger percentage value from the maximal energy intensity level tolerable by the subject, if the evaluated pain sensation level is higher than a tolerable pain sensation threshold of the subject. In some embodiments, the energy intensity is reduced to a range of 50-95%, for example 50-85%, 70-90%, 80-95% or any intermediate, smaller or larger range of values of the maximal energy intensity level tolerable by the subject. Alternatively or additionally, the duration of energy delivery is reduced in at least 5%, for example at least 10%, at least 20% or any intermediate, smaller or larger percentage value from the previous treatment duration value, if the evaluated pain sensation level is higher than a tolerable pain sensation threshold of the subject.

According to some exemplary embodiments, in order to decrease pain sensation, post cooling time duration of the skin is increased in 1-20 seconds, for example 1-5 seconds, 3-10 seconds, 5-15 seconds, 7-20 seconds or any intermediate, shorter or longer time duration. In some embodiments, cooling of the transducers, for example by the cooling module and/or by cooling a base on which the transducers are mounted, before, during and/or post energy delivery, is used to decrease pain sensation.

Reference is now made to FIG. 9D, depicting system actions when enabling treatment delivery at a selected location, according to some exemplary embodiments of the invention.

According to some exemplary embodiments, applicator and/or ultrasound transducers position is identified at block 2470. In some embodiments, the position is identified based on signals received from at least one position sensor, and/or based on signals from an optic sensor, for example optic sensor 111 shown in FIG. 1, connected to the ultrasound system or that is an integral component of the ultrasound system. In some embodiments, the camera photographs the head and/or an upper body of the subject of a patient and the applicator, for example to determine a relative or an absolute position of the applicator relative to one or more treatment targets in the face and/or neck. In some embodiments, an orientation of the applicator is identified based on signals received from at least one orientation sensor, for example, an accelerometer.

According to some exemplary embodiments, signals received from the camera and/or a position sensor of the applicator, for example sensor 115 shown in FIG. 1, are used to determine whether the applicator is located in an undesired region of the face or neck. Alternatively or additionally, signals received from the camera and/or the position sensor allow, for example, to direct a user of the system to a previously treated location, for example to repeat the treatment at a specific treatment target.

According to some exemplary embodiments, a control circuitry of the ultrasound system determines if the identified position is an allowed position at block 2472. In some embodiments, the control circuitry determines if a position of the applicator is allowed by determining a relation between the identified position of the applicator and known allowed and/or not allowed positions stored in a memory of the ultrasound system.

According to some exemplary embodiments, if an identified position is not an allowed position, then an alert indication, for example an alert signal is delivered at block 2478 by the ultrasound system. In some embodiments, the alert signal is a human detectable signal, for example an audio and/or a visual signal.

According to some exemplary embodiments, if an identified position is not an allowed position, then the ultrasound system suggests an alternative position, at block 2480.

According to some exemplary embodiments, if the position is an allowed position, then energy delivery to the ultrasound transducers and/or from the ultrasound transducers is unlocked, at block 2474. In some embodiments, the energy delivery is unlocked by allowing activation of the ultrasound transducers.

According to some exemplary embodiments, if the position is an allowed position, and optionally if energy delivery is allowed, an indication is delivered to the user of the ultrasound system at block 2476. for example a human detectable indication.

### Exemplary Information Flow

Reference is now made to FIG. 9E depicting information flow to and from the ultrasound system, according to some exemplary embodiments of the invention.

According to some exemplary embodiments, the ultrasound system 2486 collects information from a subject 2490, for example as described in FIGS. 9B and 9C. In some embodiments, at least some of the collected information is stored and/or processed in the ultrasound system 2486. Additionally, at least some of the information collected from the subject and/or treatment-related information for example at least one treatment protocol and/or values of at least one treatment parameter, is stored and/or processed in a remote device 2492, for example a remote computer, a remote server, a cloud storage or any remote device that is configured to store and/or process information.

According to some exemplary embodiments, the remote device stores at least some of information received from the ultrasound system as part of a database, for example a big data database. In some embodiments, the remote device stores at least one algorithm or a lookup table to allow processing of the stored information.

According to some exemplary embodiments, at least some of the information collected by the ultrasound system is inserted by a user 2488 of the system, for example a technician or a physician or any person qualified to operate the ultrasound system. In some embodiments, the user 2488 receives information from the ultrasound transducer, for example suggested treatment protocols and/or treatment parameter values. In some embodiments, the treatment parameter values and/or at least one treatment protocol are stored in the remote device. In some embodiments, the ultrasound system 2486 receives information requested by the user 2488 from the remote device 2492.

According to some exemplary embodiments, the ultrasound system 2486 is configured to transmit information to the remote device during and/or following treatment delivery. In some embodiments, the information transmitted to the remote device includes feedback information, for example evaluation data, on the treatment delivered to the subject. In some embodiments, the received feedback data is used to update at least one protocol stored in the remote device and/or parameters thereof. Alternatively, the received feedback data is used to generate at least one new protocol, and/or to provide suggestions to the user 2488 how to modify the treatment.

### Exemplary Validation Studies

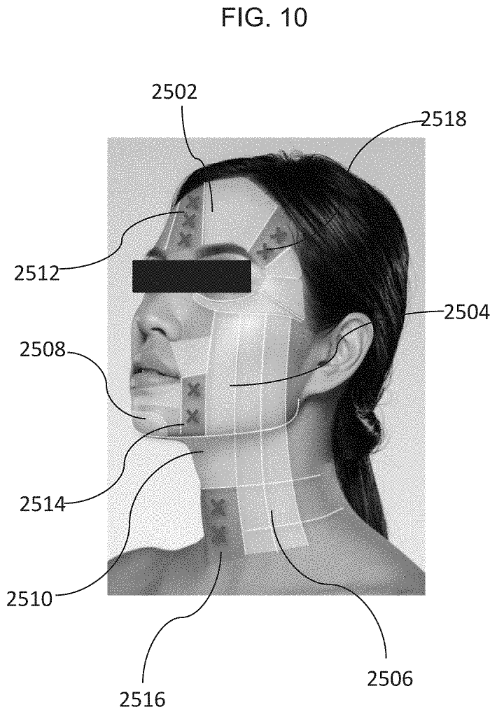

Different clinical studies were performed for treating facial and submental regions using ultrasonic energy. In these clinical trials and in some embodiments, the treated skin regions include, as shown in FIG. 10 showing a left side of a subject's face, at least one of the following: right and left side of the face, for example region 2504; regions above the right and left brows, for example region 2502; the chin area, for example region 2508; and the left side and the right side of the upper neck, for example regions 2506 and 2510. A region of the Chin beneath the mandibula, for example region 2510, is the submental region. It should be noted that in some embodiments of the invention one or more of the indicated regions are treated. Alternatively, in some embodiments of the invention, other facial and/or neck regions are treated.

In the clinical trials and in some embodiments of the invention, facial and/or neck areas that include facial nerves that pass close to the skin external surface, for example nerves located at a distance of up to 5 cm from an external surface of the skin, for example up to 3 cm, up to 1 cm or any intermediate, smaller or larger distance from the external surface of the skin, were not treated. For example region 2512 which is located above the nose, region 2514 located between the lower jaw and the side of the mouth, and region 2516 located at the center bottom of the neck. It should be noted that in some embodiments of the invention other facial and/or neck regions are not treated.

According to some exemplary embodiments, for example as described in FIG. 9D the nerves are detected using one or more imaging techniques, for example ultrasound energy. In some embodiments, the nerves are detected based on an echo received by one or more transducers of the applicator. In some embodiments, the nerves are detected or a site that should not be treated is detected when detecting an elongated element within the body which is parallel to the vertical axis of the body.

In both clinical trials, each of the treated regions received ultrasonic energy in a series of pulses, each pulse with an energy of 2-5 Joules. The series of pulses were delivered to each of the regions in two repetitions. In addition, each of the treated regions was pre and post cooled for time duration of 1 second. The table below summarizes the parameter values that were used in the trials for each treated region:

| | | | | Right side | Left side |
|---|---|---|---|---|---|
| | Right side | Left side | Right and | of the | of the |
| | of the Face¹ | of the Face | Left Brows | Upper Neck | Upper Neck |
| Pulse Energy [J] | 3.94 ± 0.38 | 3.92 ± 0.39 | 3.85 ± 0.43 | 3.91 ± 0.38 | 3.89 ± 0.39 |
| | [2.8-5] | [2.8-5] | [2.4-5] | [2.8-5] | [2.8-5] |
| Number of passes | 2² | 2² | 2² | 2² | 2² |
| Number of pulses | 35 ± 11 | 31 ± 9 | 27 ± 11 | 31 ± 11 | 23 ± 7 |
| | [10-56] | [10-50] | [8-70] | [11-68] | [7-40] |

It should be noted that in some embodiments of the invention, a different number of pulses, a different energy levels per pulse, a different number of passes per treated region and/or a different cooling duration pre and post treatment, are used.

### Clinical Study No. 1

30 subjects having a Fitzpatrick Wrinkle Score (FWS) of IV-VI were enrolled in the study. The age of the subjects was in a range of 48-61. The race distribution of the enrolled subjects was as follows: 80% Caucasian (24 subjects), 10% African American (3 subjects), 7% Asian (2 subjects), and 3% other (1 subject). The skin phototype of 90% the subjects was II, III and IV. The FWES baseline of the subjects was IV-VII. It should be noted that in some embodiments of the invention different inclusion criteria and/or a different profile of subjects is used.

In the clinical study, the subjects were pre-treated with a mixed solution of lidocaine and tetracaine (30% lidocaine/7% tetracaine). The treatment was delivered to the full face and anterior neck: left face, right face, forehead, left neck/submental, right neck/submental. The treatment parameters were as described in the upper table. It should be noted that in some embodiments of the invention, a different pretreatment is performed, different analgesic/anesthetizing agents are used, different regions are treated and/or different treatment parameters are used.

In the clinical study, a standard 35 mm photography was taken before the treatment, at 1 week and 3 months (12 weeks) following the treatment. At the 12 weeks follow up visit, a change is FWS scale and an improvement in a global aesthetic improvement scale (GAIS) is determined. In addition, a subject evaluation of improvement and satisfaction was examined. It should be noted that in some embodiments of the invention different time periods for evaluation of treatment efficacy, and different evaluation scales are used.

Safety results: 90% developed Erythema and 33% developed Edema as an immediate response to the treatment. Both the erythema and edema were resolved within 1 hour. The average pain level was 8.3 out of 10. No other adverse events (pigment alteration, vesiculation scarring) were demonstrated.

The clinical study results at the 12 weeks follow up visit demonstrated that 87% of the subjects (26 subjects) had a −1, −2 or −3 reduction in FWES score, as shown in the table below. 13% of the subjects (4 subjects) showed no improvement. The average reduction in FWES was −1.03 (p<0.0005). The clinical condition, for example reduction of wrinkles, of 87% of the subjects were improved or significantly improved.

| Δ FWES Score | Number of | Rate |
|---|---|---|
| Baseline to 12 w | Subjects | % |
| 0 | 4 | 13.3% |
| −1 | 22 | 73.3% |
| −2 | 3 | 10% |
| −3 | 1 | 3.3% |
| Total | 30 | 100% |

The subject assessment summary at the 12 weeks follow up visit demonstrated that 67% of the subjects ranked the results as improved or significantly improved. None of the enrolled subjects ranked their change as worse. 43% of the subjects were satisfied or very satisfied with the improvement. 37% of the subjects had no opinion. 20% of the subjects were unsatisfied and 1 subject was very unsatisfied.

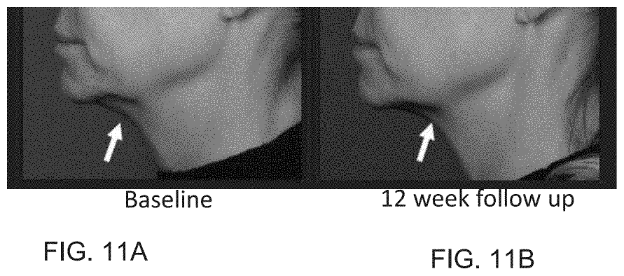

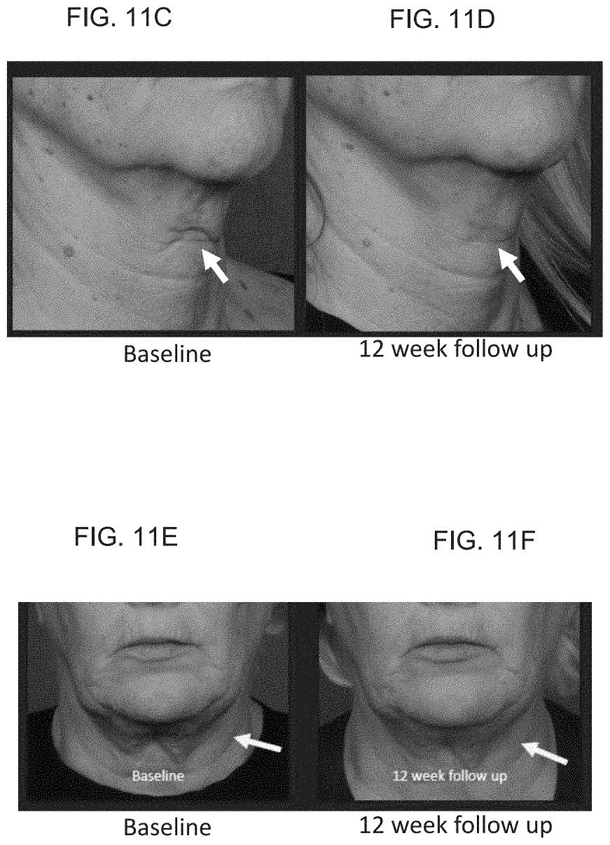

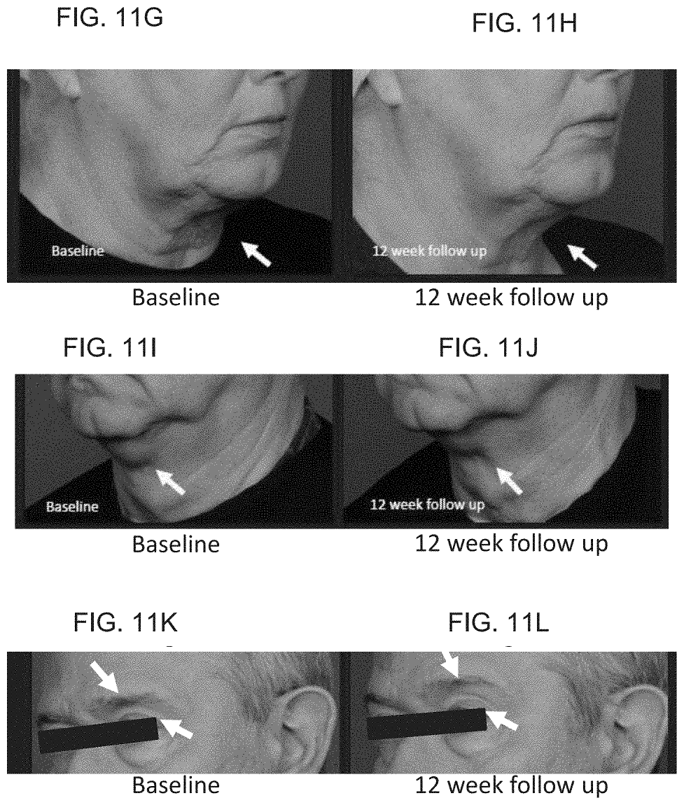

FIGS. 11A-11J show a decrease in submental fullness, 12 weeks following the treatment compared to a baseline image. FIGS. 11E-11J show a decrease in anterior neck wrinkles. FIGS. 11K and 11L show brow elevation and an increased upper lid show.

### Clinical Study No. 2

30 subjects were enrolled. 90% of the enrolled subjects were females (26 subjects) and 10% were male (3). One subject was excluded from the study due to reasons not related to the study. The race distribution of the enrolled subjects was as follows: 83% Caucasian (24 subjects), 7% African American (2 subjects), 7% Asian (1 subject), and 7% other (2 subject). The skin phototype of 93% the subjects was II-IV. It should be noted that in some embodiments of the invention different inclusion criteria and/or a different profile of subjects is used.

The treatment was delivered to the full face and anterior neck: left face, right face, brows, left neck, right neck+mid-submental. The treatment parameters were as described in the upper table. It should be noted that in some embodiments of the invention, a different pretreatment is performed, different analgesic/anesthetizing agents are used, different regions are treated and/or different treatment parameters are used.

Safety results: Erythema was observed among 79% of the subjects, and Edema in 48% of the subjects. Both the erythema and edema were resolved within 1 hour. The average pain level was 6.6 out of 10. No adverse events were demonstrated throughout the study duration.

The clinical study results at the 12 weeks follow up visit, as shown in the below table, demonstrated that 86% of the subjects were improved by either −1 or −2 Elastosis Scale (ES) scores. 14% of the subjects (4 subjects) did not show an improvement in the ES score. An average reduction of -1.07 (P<0.0005) was demonstrated 12 weeks following the treatment. 89% of the subjects were ranked as improved, significantly improved or very much improved.

| Delta ES Score | Number of | Rate |
|---|---|---|
| (12 Week-Baseline) | Subjects | % |
| 0 | 4 | 14% |
| −1 | 18 | 64% |
| −2 | 6 | 22% |
| Total | 28 | 100% |

The subject assessment summary at the 3 months follow up visit demonstrated that 78% of the subjects ranked their results as improved or significantly improved. Only one subject ranked his score as being worse. 75% of the subjects felt either satisfied or very satisfied with their results, 14% had no opinion and 11% felt unsatisfied (2 subjects) or very unsatisfied (1 subject).

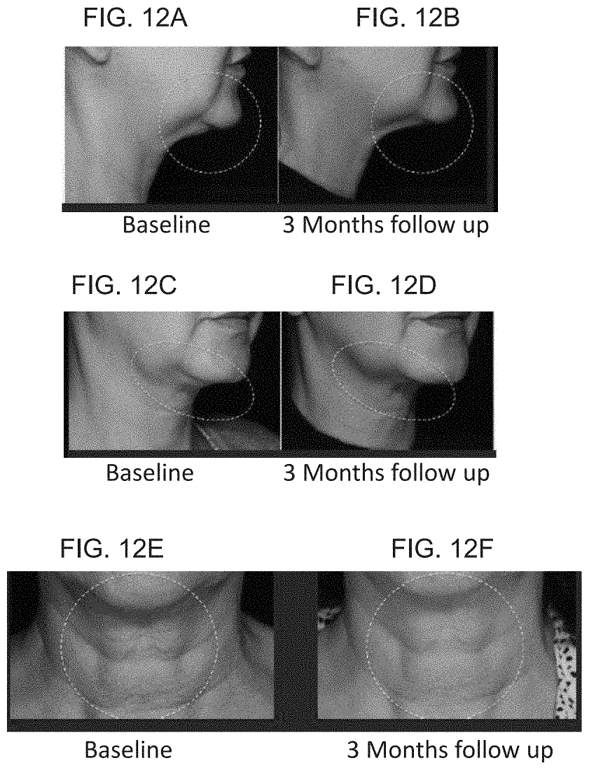

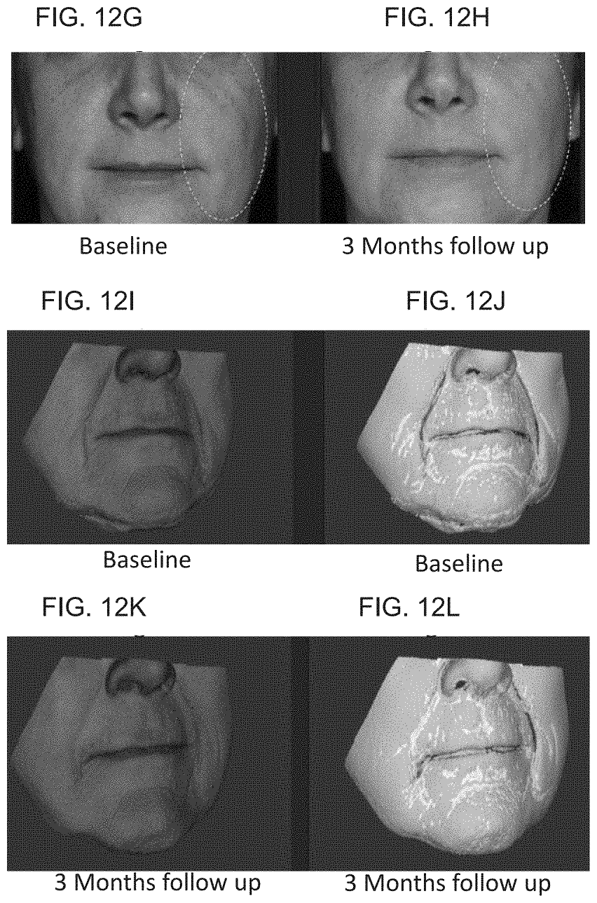

FIGS. 12A-12F show a decrease in submental fullness and neck wrinkles 3 months following the treatment compared to a baseline image. FIG. 12H shows a decrease in depth of face wrinkles at a designated area 3 months following the treatment, compared to a baseline image 12G. FIG. 12L is a 3D model imaging analysis of FIG. 12K taken 3 months following the treatment, that shown reduction in depth of facial wrinkles compared to a baseline image 12I and a 3D model imaging analysis (FIG. 12I) of the baseline image. The reduction in the wrinkles depth is demonstrated by the shift in color from red to yellow.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

As will be appreciated by one skilled in the art, aspects of the present invention may be embodied as a system, method or computer program product.

Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system”.

Furthermore, aspects of the present invention may take the form of a computer program product embodied in one or more computer readable medium(s) having computer readable program code embodied thereon. Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing.

More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. (canceled)

2. A method for treating skin, comprising:
delivering one or more pulses of non-converging ultrasonic energy to one or more skin regions through a surface area size in a range of 3 mm²-7 mm², wherein each pulse having an intensity in a range of 15 W/cm²-30W/cm² and a time duration in which said ultrasonic energy is actively transmitted in a range of 1-10 seconds per pulse;
wherein said delivering comprises delivering said each pulse with a total energy level in a range between 2-5 Joules.

3. A method according to claim 2, wherein said delivering comprises delivering said each pulse with a total energy level selected, so any generated edema and/or erythema in said one or more skin regions is resolved within 1 hour following said delivering.

4. A method according to claim 2, comprising cooling an external surface of said one or more skin regions for a time period of 1-10 seconds following stopping of said delivering.

5. A method according to claim 4, comprising:
selecting at least one of, said intensity, said time duration for transmitting said ultrasonic energy, and a time duration of said cooling according to one or more parameters of said skin region.

6. A method according to claim 5, wherein said one or more parameters of said skin region comprises one or more of, tissue type of said skin, tissue composition in said skin region, fat tissue content in said skin region, location of said skin region, presence of nerves in or near said skin region.

7. A method according to claim 2, wherein said delivering comprises delivering said one or more pulses of non-converging ultrasonic energy to said one or more skin regions located in facial and/or neck regions.

8. A method according to claim 2 comprising adjusting a number of passes of an ultrasound transducer transmitting said one or more pulses of non-converging ultrasonic energy during said delivering, over said one or more skin regions according to a location of said one or more skin regions, wherein in each pass said one or more pulses of non-converging ultrasonic energy is delivered to said one or more skin regions.

9. A method according to claim 2, comprising:
identifying one or more regions in said skin area which include one or more nerves located at a depth in a range of 0.5 mm-3 mm from an external surface of the skin prior to said delivering, and wherein said delivering comprises delivering said non-converging ultrasonic energy to a skin area that do not include said one or more regions.

10. A method according to claim 2, wherein said delivering comprises delivering said one or more pulses of non-converging ultrasonic energy to said one or more skin regions in at least two repetitions with a time difference of at least 30 seconds between the repetitions.

11. A method according to claim 2, wherein said delivering comprises delivering said non-converging ultrasonic energy for a time period long enough to have a reduction of at least one point in a wrinkles severity scale comprising one or more of the Wrinkle Severity Rating Scale (WSRS), the Glogau scale, the Fitzpatrick wrinkle scale, the Fitzpatrick wrinkle score, and/or the Fitzpatrick Wrinkle and Elastosis Scale (FWES), at least a week after said delivering.

12. A method according to claim 2, comprising heating by said delivered non-converging ultrasonic energy one or more tissue layers located at a depth of 0.25 mm-5 mm from an external surface of the skin to a temperature of at least 45 degrees Celsius.

13. A system for treating wrinkles, comprising:
an ultrasound applicator comprising;
a plurality of ultrasound transducers configured to generate non-converging ultrasonic energy, wherein each of said ultrasound transducers comprises an active surface with an area surface size in a range of 3 mm²-7 mm²;
housing shaped and sized to place at least some of said ultrasound transducers in contact with an external surface of skin tissue;
a control console connected to said ultrasound applicator, comprising:
a memory for storing parameter values of said ultrasonic energy;
a control circuitry configured to signal said ultrasound transducers to generate and deliver one or more pulses of said non-converging ultrasonic energy to one or more skin regions, wherein each of said one or more pulses has an energy intensity in a range of 15 W/cm2-30 W/cm² and a time duration in which said ultrasonic energy is actively transmitted in a range of 1-10 seconds per pulse,
and wherein said control circuitry is configured to signal said ultrasound transducers to generate each pulse with a total energy level in a range between 2-5 Joules.

14. A system according to claim 13, wherein said control circuitry is configured to signal said ultrasound transducer to generate each of said one or more pulses with intensity and a time duration selected so any generated edema and/or erythema in said one or more skin regions is resolved within 1 hour following said deliver of said one or more pulses.

15. A system according to claim 13, wherein said applicator comprises a cooling module configured to cool said plurality of ultrasound transducers and/or regions of said applicator placed in contact with the skin, wherein said control circuitry signals said cooling module to cool said plurality of ultrasound transducers and/or regions of said applicator placed in contact with the skin, for a time period of 1-10 seconds following stopping of the generation and the delivery of said one or more pulses.

16. A system according to claim 13, wherein said memory stores indications of skin tissue properties at a target region, and values of at least one treatment parameter of an unfocused ultrasound treatment, and wherein said control circuitry automatically adjusts said stored values of said at least one treatment parameter based on said stored skin tissue properties indications.

17. A system according to claim 16, wherein said skin tissue properties comprise location of said treatment target, location of at least one blood vessel and/or location of at least one nerve in said target region.

18. A system according to claim 16, wherein said tissue properties comprise wrinkles length, wrinkles depth, and/or wrinkles density at said target region.

19. A system according to claim 16, wherein said at least one treatment parameter comprises said energy intensity of each pulse, said duration of each pulse, number of transducers, temperature and/or duration of skin surface cooling.

20. A system according to claim 13, wherein said memory stores indication related to pain sensitivity of a subject and wherein said control circuitry reduces ultrasound intensity in at least 5% and/or increases increasing post cooling time duration in at least 5% base on said stored pain sensitivity indications.
